(19) United States
(12) Patent Application Publication
(10) Pub. No.: US 2023/0277774 A1
(43) Pub. Date: Sep. 7, 2023

(54) ELECTRONIC SYSTEM FOR A DRUG DELIVERY DEVICE AND DRUG DELIVERY DEVICE

(71) Applicant: Sanofi, PARIS (FR)

(72) Inventors: Stefan Alt, Frankfurt am Main (DE); Oliver Charles Gazeley, Basel (CH); Adam Moyo Harvey-Cook, Billericay, Essex (GB); Aiden Michael O'Hare, Warwick, Warwickshire (GB); Stephen Ingram, Warwick, Warwickshire (GB); Paul Andrew Calton, Warwick, Warwickshire (GB)

(21) Appl. No.: 18/016,995

(22) PCT Filed: Jul. 21, 2021
(86) PCT No.: PCT/EP2021/070314

§ 371 Date: Jan. 19, 2023

(30) Foreign Application Priority Data
Jul. 23, 2020 (EP) 20315355.6

(51) Int. Cl. A61M 5/315

(52) U.S. Cl. CPC A61M 5/31546; A61M 2205/505

(57) ABSTRACT

An electronic system for a drug delivery device is provided. The electronic system comprises: at least one user interface member, which is arranged to be manipulated by a user of the drug delivery device, and an electrical user proximity detection unit. The user interface member has an exterior operation surface arranged and configured to be touched by a user of the drug delivery device during operation of the drug delivery device. The electrical user proximity detection unit is configured to generate a signal when the user is close to or touches the exterior operation surface.

### CROSS-REFERENCE TO RELATED APPLICATIONS

The present application is the national stage entry of International Patent Application No. PCT/EP2021/070314, filed on Jul. 21, 2021, and claims priority to Application No. EP 20315355.6, filed on Jul. 23, 2020, the disclosures of which are incorporated herein by reference.

### TECHNICAL FIELD

The present disclosure relates to an electronic system for a drug delivery device. The present disclosure further relates to a drug delivery device, which preferably comprises the electronic system.

### BACKGROUND

Drug delivery devices using electronics are becoming increasingly popular in the pharmaceutical industry as well as for users or patients. However, especially if the device is designed to be self-contained, that is to say without a connector for a connection to an external electrical power source which is necessary to provide electrical power for the operation of the device, the management of the resources of a power supply integrated into the device is particularly important.

### SUMMARY

The present disclosure provides improvements for drug delivery devices comprising an electronic system or improvements for electronic systems for drug delivery devices.

One aspect of the present disclosure relates to an electronic system for a drug delivery device. Another aspect of the present disclosure relates to a drug delivery device, especially one comprising the electronic system. Accordingly, the features, which are disclosed in relation to the drug delivery device or units thereof or therefore, do also apply for the electronic system and vice versa.

In one embodiment, the electronic system comprises at least one user interface member. The user interface member may be arranged or provided to be manipulated by the user of the drug delivery device, e.g. for setting a dose to be delivered by the device in a dose setting operation and/or for delivering the set dose from the drug delivery device in a dose delivery operation.

In one embodiment, the electronic system comprises an electronic control unit. The electronic control unit may be configured to control an operation of the electronic system. The electronic control unit may be or may comprise an electronic processor, such as a microcontroller or an ASIC, for example. The electronic system may have a first state and a second state, e.g. when it is operative. The electronic system may have an increased electrical power consumption in the second state as compared to the first state. In the first state, one or more electrical or electronic units of the electronic system may be in a sleep mode or be powered off such that they have no significant power consumption or no power consumption. For example, in the second state a motion sensing unit may be active, i.e. it can be operated, where this unit is not active, i.e. it cannot be operated, in the first state. The motion sensing unit will be described in more detail below. Alternatively or additionally, a communication unit may be inactive in the first state and active in the second state. The communication unit will be described in more detail below.

In one embodiment, the user interface member has an exterior operation surface arranged and configured to be touched by a user, e.g. by the user of the drug delivery device. The exterior operation surface may be arranged and configured to be touched during operation of the system or the device, such as the dose setting operation and/or the dose delivery operation, e.g. for initiating and/or for performing the operation. Thus, the exterior operation surface may be or may comprise a setting surface and/or a delivery surface.

In one embodiment, the electronic system comprises a, preferably electrical, user proximity detection unit. The user proximity detection unit may be configured to generate a signal, e.g. an electrical signal, when the user is close to or touches the exterior operation surface. The signal may be a proximity signal and/or a use signal (see further below). In case the operation surface is the setting surface the signal may be a setting signal. The setting signal may be indicative that the user intends to perform a dose setting operation. In case the exterior operation surface is the delivery surface the signal may be a delivery signal. The delivery signal may be indicative that the user intends to perform a dose delivery operation. The electronic system may be configured such that it can distinguish between the setting signal and the delivery signal. The signal of the user proximity detection unit may be generated in response to the user touching the exterior operation surface or the user not yet touching the exterior operation surface but being less than a predetermined distance away from the exterior operation surface. The predetermined distance may be less than or equal to one of the following values: 5 mm, 4 mm, 3 mm, 2 mm, 1 mm, 0.5 mm. The user proximity detection unit may be configured to detect whether the user is close to an exterior surface of the user interface member. The user proximity detection unit may be operatively connected to the electronic control unit. Thus, the signal may be transmitted to the electronic control unit. The electronic control unit then may control operation of the electronic system in response to the signal from the user proximity detection unit, e.g. such that the system is switched to the second state of higher power consumption in response to the signal.

In one embodiment, the electronic system is configured to consider in what sequence or at what point in time during an operation sequence of the system or the device, the signal of the user proximity detection unit is generated, and/or what kind of signal is generated, e.g. setting signal or delivery signal. If, for example, a delivery signal is generated without a setting signal preceding the delivery signal within a predetermined time, e.g. 10 seconds or less, the mode of operation of the system may be different than, if a setting signal is generated, and potentially, subsequently, a delivery signal. If the delivery signal is generated without a previous setting signal, this can be used to control the operation state of the communication unit, e.g. to transmit data from the electronic system, such as dose data, to another system (or synchronize dose data with the other system or device). In case the setting signal is detected, this can be used as a signal (see the use signal discussed further below) to cause activation of the motion sensing unit as a dose delivery operation is soon to be expected, e.g. in case the motion sensing unit is configured to monitor the delivery operation.

In one embodiment, the electronic system comprises an electrical use detection unit. The electrical use detection unit may be operatively connected to the electronic control unit. The electrical use detection unit may comprise the user proximity detection unit. The electrical use detection unit may be configured to generate a use signal, e.g. an electrical signal. The use signal may be indicative that the user intends to operate the electronic system or the drug delivery device, e.g. that the user intends to perform a dose setting operation and/or a dose delivery operation. The use signal may be generated before the user actually commences the dose setting operation or the dose delivery operation, e.g. before any parts or members of the electronic system or of the drug delivery device are moved by the user. The electronic system may be configured such that it is switched from the first state into the second state in response to the use signal, e.g. by the electronic control unit. The use signal may be generated by the user proximity detection unit. Hence, referrals to the “use signal” above and below should be considered as referrals to the “signal” or the “proximity signal” generated or caused by the user proximity detection unit and vice versa. Likewise, referrals to the “use detection unit” above and below should be considered as referrals to the “user proximity detection unit” and vice versa.

In one embodiment, the system is configured such that the use signal causes the electronic control unit to issue an activation signal to at least one further electrical or electronic unit of the system in order to switch this unit to the state of higher power consumption, e.g. to an operation state, where this unit can be operated. In a first state, before being switched to the state of higher power consumption (or second state), the unit may be inactive and/or cannot be operated to perform the desired function. The at least on further electrical or electronic unit may be or may comprise the motion sensing unit and/or the communication unit.

In one embodiment, the electronic system is configured such that switching the system from the first state into the second state—e.g. with active motion sensing and/or communication unit—is completed within a time span of less than or equal to one of the following values: 5 ms, 4 ms, 3.5 ms, 3.2 ms, 3 ms, 2.8 ms, 2.7 ms, 2.5 ms (ms: milliseconds). Alternatively or additionally, the electronic system is configured such that switching the system from the first state into the second state is completed within a time span of greater than or equal to one of the following values: 1 ms, 1.5 ms, 1.7 ms, 2.0 ms, 2.2 ms, 2.5 ms. Particularly, the time span required for switching the system from the first state to the second state may be between 1 ms and 5 ms. The time span required for switching may be the time from the generation of the use signal (where the unit preferably is not yet operational) up to the point in time when the motion sensing unit and/or the communication unit has become operational.

In one embodiment, the exterior operation surface is the setting surface of the user interface member. Thus, the use signal may be generated in response to the user being close to or touching the setting surface. The setting surface may be a side surface or a lateral surface of the user interface member. The setting surface may be arranged to be contacted by the user for conducting or initiating the dose setting operation. The dose setting operation may involve a rotational movement of the user interface member, e.g. a pure rotational movement without axial displacement. The movement of the user interface member during dose setting may be relative to a housing of the electronic system or the drug delivery device. The setting surface may be arranged to be gripped by the user with more than one finger, e.g. by the thumb and the index finger, preferably simultaneously with more than one finger. Alternatively or additionally, the exterior operation surface is the delivery surface which is arranged to be contacted for conducting or initiating the dose delivery operation. Thus, the use signal may be generated in response to the user being close to or touching the delivery surface. The dose delivery operation may involve an axial movement of the user interface member, e.g. a pure axial movement without rotation. The delivery surface may be an axially oriented surface, e.g. a proximally directed surface, of the user interface member. The delivery surface may be designed to be contacted with one finger, e.g. the thumb.

In one embodiment, the electronic system comprises one user interface member, e.g. one integral member, for the dose setting operation and the dose delivery operation or two different user interface members, where one of these members is the user interface member for dose setting and the other one is the user interface member for dose delivery. The two different members are expediently movable relative to one another, e.g. to switch between a dose setting configuration and a dose delivery configuration. If one interface member is used for dose setting and dose delivery, this interface member may have the setting surface and the delivery surface, which, preferably, are not movable relative to one another, especially not for or during dose delivery and/or not for or during dose setting. If two different user-interface members are used, the setting surface and the delivery surface may be on different members and movable relative to one another for or during dose delivery and/or for or during dose setting.

In one embodiment, the electronic system or the drug delivery device comprises a housing. For conducting the dose setting operation and/or the dose delivery operation, the user interface member may be moved relative to the housing, e.g. rotated and axially, e.g. proximally, displaced for dose setting and axially, e.g. distally, displaced for dose delivery.

In one embodiment, the user proximity detection unit is configured such that the signal, e.g. the use signal or proximity signal, is generated responsive to an event which occurs before the user interface member is moved, e.g. relative to the housing and/or for conducting the dose setting operation and/or the dose delivery operation. The event in response to which the signal is generated may be the user being close to our touching the exterior operation surface of the user interface member. Preferably, the use signal is generated prior to an event which it should be indicative for. For example, if the use signal is indicative for a dose setting operation, it is preferably generated before the user interface member is moved for the dose setting operation. If the use signal is indicative for a dose delivery operation, it is preferably generated after the dose setting operation and before commencing the dose delivery operation for delivering the set dose.

In one embodiment, the user proximity detection unit comprises an electrical sensor or switch which is arranged and configured to detect whether the user, for example the user's hand or one or more of the user's fingers, is close to the exterior operation surface or touches the exterior operation surface.

In one embodiment, the electronic system comprises an electrical power source, e.g. a battery. The power source, preferably along with further electrical or electronic components of the electronic system, e.g. all components, may be integrated into the interior of the user interface member. The power source may be provided to electrically power the electrically operated units or components of the system.

In one embodiment, the electrical sensor or switch is a contactless or touchless sensor or switch, e.g. a proximity sensor or switch. Specifically, the sensor or switch may not have any electrically conductive surfaces or contact surfaces accessible on the exterior operation surface.

In one embodiment, the electrical sensor or switch is a capacitive sensor or switch. Capacitive sensor or switches are particularly suitable to sense the user's proximity without requiring mechanical contact between the user's hand and the exterior operation surface. Capacitive sensors or switches usually operate based on the capacitance between an electrode of the sensor and the object which is to be sensed, preferably the user's finger(s).

In one embodiment, the electrical sensor or switch comprises an electrode and, preferably, a circuit operatively connect to the electrode, e.g. an integrated circuit. The electrode may define the sensing surface of the sensor. The electrode may be associated with the exterior operation surface, e.g. accessible on the surface or arranged at a distance from the surface but configured to sense or monitor events occurring on that surface or close to that surface.

In one embodiment, the electrical sensor or switch is a contact or touch sensor or switch. Such a sensor may require mechanical contact with the exterior operation surface and/or with a sensing surface of the sensor, e.g. an electrode, to generate the signal, which indicates that the user touches the exterior operation surface. In contactless or touchless sensors, such a mechanical contact may not be required.

In one embodiment, the sensor or switch is a resistive sensor or switch. Accordingly, the sensor may monitor or the switch may be triggered by the changes in the electrical resistance or resistivity within a circuit. The element changing the resistance or the resistivity may be the user's body.

In one embodiment, the sensor or switch comprises at least one electrically conductive contact surface accessible on the exterior operation surface. The contact surface may be arranged to be mechanically contacted by the user's hand or finger(s) when the user handles the user interface member. The sensor or switch may comprise a plurality of electrically conductive contact surfaces, which are accessible on the exterior operation surface. The respective contact surface may be conductively connected to an electrical power source. Two contact surfaces may be arranged such that the user, preferably with the same finger or different fingers of the same hand, may simultaneously touch both contact surfaces. The two contact surfaces may have opposite electrical polarities and/or different electrical potentials, particularly electrostatic potentials. For example, one contact surface may be conductively connected with the positive terminal of the power source and the other contact surface may be conductively connected with the negative terminal of the power source. Accordingly, if the user bridges the distance between two contact surfaces of different polarities or potentials, the current flowing through the user's hand may be used as the (use) signal as, then, it is ensured that the user touches the exterior operation surface. Having two electrically conductive contact surfaces of different electrical potential or different polarities on the exterior operation surface is one example of a resistive switch or sensor, particularly an example of a resistive touch switch or sensor. The exterior operation surface may be provided in various surface regions with pairs of contact surfaces of different potentials or polarities which are arranged to be contacted simultaneously by the user's hand. In this way, it is ensured that regardless of the surface region, e.g. on the setting surface or on the delivery surface, the (use) signal indicative for the user touching the exterior operation surface can be generated. Electrically conductive contact surfaces arranged on the exterior may further be used to charge a rechargeable power source of the electronic system.

In one embodiment, one or more contact surfaces, which are accessible on the exterior operation surface, may be recessed relative to an enveloping surface, which is defined by the (axial or radial) ends of the operation surface. In this way, the risk that conductive elements in a bag could (inadvertently) connect the two contact surfaces conductively, which could be interpreted as the (use) signal, can be reduced. The enveloping surface may be defined by connecting, e.g. by plane surface sections or by curved surface sections, the axial ends or the radial ends of the surface. The axial ends are expediently used to define the enveloping surface in case the exterior operation surface faces axially, e.g. proximally such as the delivery surface, whereas the radial ends are used for a radially facing surface, such as the setting surface.

In one embodiment, the electronic system comprises at least one of, an arbitrarily selected plurality of, or all of the following units or components:
an electrical motion sensing unit. The motion sensing unit will be explained in more detail below.
a communication unit. The communication unit may be provided to establish the communication interface between the electronic system and another device such as an electronic device such as a portable device, e.g. a portable or non-portable computer, a mobile phone or a tablet. The communication unit may be a wireless unit, e.g. an RF communication unit, such as a Bluetooth unit. The communication unit may be provided to transmit dose data from the electronic system to the other device, e.g. information on the amount of drug delivered by the device in a delivery operation.
a memory unit. The memory unit may be provided to store executable program code and/or data on dose information which has been calculated by the electronic system, preferably data on the delivered doses. The data on the doses may be determined via the motion sensing unit.

In one embodiment, the motion sensing unit is configured to generate one or more electrical motion signals. The motion signal(s) may be suitable to quantify the relative movement between a first member and a second member, e.g. during the dose setting operation or the dose delivery operation. The first member and/or the second member may be members of the electronic system and/or the drug delivery device. The relative movement may be relative rotational movement. For example, the first member may rotate relative to the second member during dose delivery.

In one embodiment, the electronic system or the drug delivery device comprises a dose setting and/or drive mechanism. The dose setting and/or drive mechanism may comprise the first member and the second member. The first member and/or the second member may be configured to move during the dose setting operation and/or the dose delivery operation relative to the housing of the electronic system or the drug delivery device. The first member may be a dose member or dial member of the dose setting and/or drive mechanism, which is moved to set a dose, e.g. a dial sleeve or a number sleeve. The second member may be a drive member, e.g. a member engaged with a piston rod of the dose setting and/or drive mechanism, or a device user interface member, such as a dose knob and/or injection button. The first member and/or the second member may be movably coupled to and/or retained in the housing. In the dose setting operation, the first member and/or the second member may be displaced axially relative to the housing, for example away from a proximal end of the housing. The distance by which the first member and/or the second member is displaced during the dose setting operation relative to the housing axially may be determined by the size of the set dose. In other words, the drug delivery device may be of the dial extension type, i.e. the device increases its length during the dose setting operation in an amount proportional to the size of the set dose.

In one embodiment, in the dose setting operation and/or in the dose delivery operation, the first member moves, e.g. rotates and/or moves axially, relative to the second member. For example, the first member may rotate relative to the second member during the dose delivery operation, e.g. only during the dose delivery operation. The first member and the second member may both move axially during the dose delivery operation. The first member may rotate relative to the second member and relative to the housing during the dose setting operation and/or the dose delivery operation. The second member may be rotationally locked or guided with respect to the housing during the dose delivery operation, e.g. by a delivery clutch. The first member and the second member may be rotationally locked relative to one another during the dose setting operation. Accordingly, the first member and the second member may rotate relative to the housing in the dose setting operation. During the dose setting operation the first member and the second member may be coupled to one another, e.g. via a coupling interface, e.g. a setting clutch. The coupling interface may rotationally lock the first member and the second member to one another during the dose setting operation. When the coupling interface is engaged, the first member and the second member may be rotationally locked with one another, such as by direct engagement of coupling interface features. The first member and the second member may comprise mating coupling interface features. The coupling interface may be released during the dose delivery operation. Hence, the second member may be rotationally locked relative to the housing during dose delivery, whereas the first member may rotate relative to the housing during dose delivery. The coupling interface may be released when switching from a dose setting configuration into a dose delivery configuration.

In one embodiment, the first member and the second member rotate relative to one another during only one of the dose setting operation and the dose delivery operation. One of the first member and the second member, e.g. the first member, may rotate relative to the housing during both operations. One of the first member and the second member, e.g. the second member, may rotate relative to the housing during only one of the operations, e.g. during dose setting or during dose delivery.

In one embodiment, the electronic system is configured such that the motion sensing unit is switched unit from a first state into the second state, e.g. by the electronic control and/or in response to the use signal. In the first state, the motion sensing unit may be not operative to sense movement of the first member relative to the second member. In the second state, the motion sensing unit may be operative. That is to say, it may be operated to sense movement of the first member relative to the second member. In the second state, the motion sensing unit may have a power consumption which is greater than in the first state. The increase in power consumption of the motion sensing unit may contribute to or define the increased power consumption of the electronic system in the second state.

In one embodiment, the motion sensing unit is configured to operate during the dose delivery operation, preferably only during the dose delivery operation. The motion sensing unit may be configured to monitor the dose delivery operation, e.g. the rotation of the first member relative to the second member. Thus, from the motion signals, positional information on the relative position between the first member and the second member can be gathered. Alternatively or additionally, it is also possible to gather positional information between two members in the dose setting operation. However, in order to calculate dose information on the dose delivery during the dose delivery operation, it is advantageous to monitor the movements during the dose delivery operation by the motion sensing unit.

In one embodiment, the electronic control unit or the electronic system is configured to calculate dose information utilizing the motion signals generated by the motion sensing unit. As noted previously, the dose information preferably is information on the size of the dose which is delivered in the dose delivery operation.

In one embodiment, during the dose setting operation, the dose may be set, e.g. between a minimum settable dose and a maximum settable dose. The dose may be set, preferably in quantities corresponding to whole-number multiples of one unit dosage increment.

In one embodiment, the exterior operation surface, in particular the surface monitored to generate the use signal, is the setting surface. Thus, the use signal may be indicative for the user being close to or touching the setting surface. In this case, the use signal may be a setting signal.

In one embodiment, the exterior operation surface, in particular the surface monitored to generate the use signal, is the delivery surface. Thus, the use signal may be indicative for the user being close to or touching the delivery surface. In this case, the use signal may be a delivery signal.

Using a setting signal as the use signal has the advantage that, as the dose has to be set before it is delivered, there is more time for switching the motion sensing unit to the second state. Using a delivery signal as the use signal has the advantage that setting events which occur without a subsequent dose being dispensed do not drain power from the system. Regardless of whether the use signal is the setting signal or the delivery signal, the motion sensing unit preferably monitors movement during the dose delivery operation.

In one embodiment, the motion sensing unit comprises an optoelectronic emitter and preferably an optoelectronic detector.

In one embodiment, the motion sensing unit is configured to generate (electrical) motion signals. The motion signal(s) may be suitable to quantify relative rotational movement between the first member and the second member, especially during a dose setting operation for setting a dose to be delivered by the drug delivery device, e.g. a dose of drug, and/or during a dose delivery operation for delivering the set dose. The motion sensing unit may comprise one or more sensors and/or one or more emitters, e.g. one or more optoelectronic radiation sensors or detectors and/or one or more optoelectronic radiation emitters. The electronic system, particularly the motion sensing unit, may be configured to generate motion signal(s) in response to movement of the first member relative to the second member. Dose data may be calculated from the motion signals, e.g. on the dose which is delivered during the dose delivery operation.

In one embodiment, the electronic system comprises a timer unit. The timer unit may be configured to deactivate the motion sensing unit and/or other electrically powered units of the electronic system after a predetermined time period has elapsed and, preferably when in this time period no motion signal and/or no use signal is generated. The timer unit may trigger or cause the electronic system to be switched from the second state back to the first state. In other words, the electronic system may be configured to switch from the second state back to the first state, preferably when for a predetermined time no motion signal is generated and/or received by the electronic control unit.

In one embodiment, the electronic system comprises a wake-up unit. The wake-up unit may be configured to wake up the electrical use detection unit and/or the user proximity detection unit. The wake-up unit may be configured to generate an electrical wake-up signal. The electronic system may be configured to switch the electrical use detection unit and/or the user proximity detection unit from a first state into a second state, particularly in response to the wake-up signal. The first state may be a state in which the respective detection unit is not operative. The second state may be a state in which the respective detection unit is operative. The power consumption of the detection unit in the first state may be lower than the power consumption in the second state.

In one embodiment, the wake-up unit comprises an electrical wake-up sensor or switch, which is configured to generate the wake-up signal. The wake-up unit may be or may comprise a user proximity detection unit, particularly an additional user proximity detection unit to the one discussed above which may be part of the use detection unit. Thus, the wake-up signal may be generated in response to the user's proximity, e.g. to a surface of the user interface member such as the setting surface, for example. The wake-up sensor or switch may be a proximity sensor or switch. Alternatively, different sensing technologies than proximity sensing may be used in order to wake the respective detection unit such as vibration sensing.

In one embodiment, the wake-up sensor or switch is a vibration sensor or switch. Vibrations detected by the sensor or switch may be characteristic for preparatory movements of a user preparing the electronic system or the device for a dose setting operation or a dose delivery operation. Especially, during storage of the electronic system vibrations do not tend to occur often. Thus, power drain during storage caused by non-necessary sensing operations of the user proximity detection unit may be prevented or at least reduced by a vibration sensor. An orientation sensor may also be suitable as wake-up sensor.

In one embodiment, the power consumption of the use detection unit or user proximity detection unit in the first state of the electronic system, e.g. the state where the motion sensing unit and/or the communication unit is not active, is less than the one of the motion sensing unit, the communication unit and/or the electronic system in the second state of the electronic system. In the first state, the power consumption of the motion sensing unit may be less than the one of the use detection unit or user proximity detection unit in the same state.

In one embodiment, the power consumption of the wake-up unit is smaller than the power consumption of the use detection unit or the user proximity detection unit, when the respective unit is active or operable. Accordingly, the wake-up unit may contribute to using our resources efficiently.

In one embodiment, the wake-up sensor or switch is a capacitive sensor or switch. Alternatively or additionally, the wake-up sensor or switch may comprise a resistive sensor or switch. This is particularly suitable, if a user proximity detection unit is used in the wake-up unit.

In one embodiment, in case a capacitive sensor or switch is used for the wake-up unit, and a capacitive sensor is used for the use detection unit or user proximity detection unit, the respective sensors may be configured to operate in different frequencies. That is to say reading events or sensing events for the wake-up unit may occur with a lower frequency than reading events or sensing events for the use detection unit or the user proximity detection unit. This is particularly advantageous, in case the wake-up unit monitors proximity of the user to the setting surface and the user proximity detection unit monitors proximity of the user to the delivery surface of the user interface member. In other words, a frequency with which the wake-up sensor is operated may be less than the one with which a (proximity) sensor or switch of the use detection unit is operated. Higher frequencies ensure earlier detection of relevant events, whereas lower frequencies save electrical power.

In one embodiment, a user proximity detection unit of the wake-up unit uses the setting surface as exterior operation surface. Alternatively or additionally, the user proximity detection unit for the use detection unit uses the delivery surface as exterior operation surface.

In one embodiment, the setting signal is the use signal.

In one embodiment, the delivery signal is the use signal.

In one embodiment, the electronic system is configured such that the delivery detection unit can be operated in two states, a first state of low power consumption and a second state of higher power consumption. The electronic control unit may be configured to switch the delivery detection unit into the state of higher power consumption in response to the setting signal. Hence, the setting signal may be the wake-up signal. The electronic control unit may be configured to switch the electronic system into the second state in response to the delivery signal, which, accordingly, may be the use signal.

In one embodiment, the capacitive sensor comprises a sensor electrode and a sensor circuit operatively connected, e.g. conductively connected, to the sensor electrode. The sensor electrode may be associated with the exterior operation surface.

In one embodiment, in the second state of the electronic system, all electronic or electrical units are in an operational state and can be operated.

In one embodiment, the electronic control unit is configured to activate the delivery detection unit only after the setting signal has been generated.

In one embodiment, the electronic system is integrated into the interior of the user interface member. The user interface member may be configured to be attached to a member of a drug delivery device unit, e.g. to form a drug delivery device.

In one embodiment, the user interface member is a dose setting and/or an injection button of or for the drug delivery device.

In one embodiment, the user proximity detection unit is configured to detect the proximity of the user to the exterior operation surface, preferably without involving moving mechanical parts.

In one embodiment, the electronic system is configured such that the use signal is generated before the user interface member is moved relative to the housing for performing the dose setting operation and/or the dose dispensing operation. Specifically, the setting signal may be generated before the setting operation is commenced. The delivery signal may be generated before the delivery operation is being commenced, for example, in case a setting operation precedes the delivery signal, after the setting signal has been generated.

In one embodiment, the drug delivery device comprises a reservoir retainer for retaining a reservoir with drug, e.g. a cartridge, and/or the device comprises the reservoir. The reservoir may comprise drug sufficient for a plurality of, preferably user-settable doses, to be delivered by the drug delivery device.

In one embodiment, the drug delivery device is a pen-type device.

In one embodiment, the drug delivery device is a variable dose device. The size of the dose to be delivered by the device may be varied by the user.

In one embodiment, the electronic system is configured as a, preferably reusable, add-on for a drug delivery device unit. The system may be configured to be attached to the drug delivery device unit, e.g. clippable to the device unit. That is to say, the electronic system may be configured to be used with a plurality of drug delivery device units. The respective drug delivery device unit may be a disposable drug delivery device unit and/or the respective drug delivery device unit may be fully operational for performing dose setting operations and dose delivery operations. The drug delivery device unit may comprise the reservoir. The drug delivery device unit may be free of electrically operated units or components for such units.

In one embodiment, a kit for a drug delivery device comprises the drug delivery device unit and the electronic system. The system may be attachable to the device unit to form the drug delivery device. Features disclosed above and below for the drug delivery device, especially the ones that are not directly related to the electronic system, should also apply for the drug delivery device unit and vice versa.

In one embodiment, the electronic system comprises a connection detection unit, e.g. an electrical connection detection unit. Such a connection detection unit is particularly suitable, if the electronic system is designed to be connected to the drug delivery device unit, e.g. permanently or releasably attached to the drug delivery device unit. When the connection to the drug delivery device unit is detected by the connection detection unit, the electronic system is preferably configured to change the state of the electronic system, e.g. from non-operational to operational. The connection detection unit may be operatively connected to one or more further electrical and/or electronic units of the electronic system. The connection detection unit may be configured to generate a connection signal in response to connection of the electronic system to the drug delivery device unit. For example, the connection detection unit may comprise a switch which is triggered when the electronic system such as the user interface member thereof is attached to the drug delivery device unit in order to form the drug delivery device. The connection signal may be transmitted to one or more further units of the electronic system, e.g. the electronic control unit, the user proximity detection unit or the use detection unit. The electronic system may be configured such that, in response to the connection signal, the power consumption of the electronic system is increased, e.g. from below 1 μA to a greater value. The system may be switched to the state of increased power consumption by the electronic control unit. When the connection to the drug delivery device unit has been confirmed, the use detection unit, the user proximity detection unit and/or the wake-up unit may be activated or operable, for example. The motion sensing unit and/or the communication unit may still be inactive or not operable. The connection detection unit is particularly suitable for electronic systems which are configured as a separate module or add-on to be connected to a drug delivery device unit, as, during storage of the stand-alone system, the power consumption is advantageously small, since the connection detection unit does not indicate a connection to the drug delivery device unit. In response to the connection signal, the electronic system may change its state of operation, e.g. from an idle or powered off state to the state where further electronic or electrical operations can be performed by the system, such as a wake-up operation or a use detection operation or a user proximity detection operation. In the state before the connection signal has been generated, such an operation may not have been possible. Specifically, any electrical or electronic operation of the electronic system may require that the electronic system is connected to a drug delivery device unit, where this connection is detected by the connection detection unit. Thus, the connection detection unit may have to confirm that the system is connected to a drug delivery device unit before the system is rendered operational.

In one embodiment, the electronic system, particularly the user proximity detection unit, comprises a setting sensor and/or a delivery sensor. The respective sensor may be a proximity sensor, preferably a capacitive sensor. The system may comprise just the setting sensor or just the delivery sensor or both sensors. The setting sensor may be arranged to monitor the setting surface of the user interface member, e.g. for proximity or touch events occurring at this surface. Occurrence of the event may be indicated by the setting signal. The delivery sensor may be arranged to monitor the delivery surface of the user interface member, e.g. for proximity or touch events occurring at this surface. Occurrence of the event may be indicated by the delivery signal. The respective sensor may be a contact-based sensor or a contactless sensor, e.g. a proximity sensor such as a capacitive sensor. The setting sensor and the delivery sensor may be formed by one combined sensor. The combined sensor may not distinguish whether the user is close to or touches the setting surface or the delivery surface. Alternatively, separate setting and delivery sensors or separate sensor channels in one sensor or sensor chip may be provided. In this case, a distinction which one of the setting and delivery surfaces the user is close to or touches can be made. Each of the sensors may be operatively connected to the electronic control unit. The setting sensor may be part of the wake-up unit, e.g. the wake-up sensor, or be part of the use detection unit, e.g. to generate the use signal. The delivery sensor may be part of the use detection unit, wherein, preferably, the setting sensor is part of the wake-up unit. In case of a combined setting and delivery sensor, the sensor may be part of the wake-up unit, e.g. as wake-up sensor, and of the use detection unit, e.g. to generate the use signal, wherein, preferably, the power consumption of the combined sensor or its mode of operation is different in the functionalities for the different units. Expediently, the power consumption of the sensor in its function in the wake-up unit may be lower than in its function in the use detection unit, e.g. due to a lower response rate (see also the description further above).

In one embodiment, the user proximity detection unit is configured to provide or generate a release signal when the user releases the exterior operation surface, e.g. after it has been touched. This enables to detect the point in time when the user releases the user interface member, e.g. the setting surface or the delivery surface. In this way, it can be detected when the dose setting operation or the dose delivery operation is (likely) completed. This can be achieved by the setting sensor for the setting surface or the delivery sensor for the delivery surface based on a signal (change), which preferably is characteristic for a release of a previously touched surface.

In one embodiment, the electronic system is configured such that at least one sensor or switch of the electronic system, preferably a sensor or switch of the user proximity detection unit, e.g. the setting sensor, is deactivated or switched off in response to the signal generated by the user proximity detection unit, e.g. the setting signal. The sensor or switch may be deactivated by the electronic control unit. The deactivation reduces the power consumption of the system. However, the sensor or switch may not be available for detecting setting operations subsequent to its deactivation. Hence, it may be reactivated in response to another signal, e.g. a signal generated by the user proximity detection unit, e.g. by another sensor thereof, or by the motion sensing unit or after expiration of a timer.

In one embodiment, the electronic system is configured such that the response rate of at least one sensor or switch of the electronic system, preferably a sensor or switch of the user proximity detection unit, is changed in response to the signal generated by the user proximity detection unit. The change may be performed by the electronic control unit issuing according instructions. The sensor or switch may be the setting sensor and/or the delivery sensor. The response rate may be the rate or frequency with which the sensor performs measurements or with which it is operated or polled, e.g. by the electronic control unit. Before and after the change the response rate is expediently greater than zero.

In one embodiment, the user proximity detection unit comprises a plurality of sensors or switches, e.g. the setting sensor and the delivery sensor. The electronic system may be configured such that the response rate of one sensor or switch, e.g. to touch or proximity events, of the user proximity detection unit is changed in response to a signal generated by another sensor or switch of the user proximity detection unit. For example, the response rate of the at least one sensor or switch is increased in response to the signal generated by the user proximity detection unit. Alternatively, the response rate of the at least one sensor or switch is decreased in response to the signal generated by the user proximity detection unit. For example, the response rate of the delivery sensor may be changed, e.g. increased, in response to the setting signal by the setting sensor (which is indicative for the user touching the setting surface). The response rate of the setting sensor may be changed, e.g. decreased, in response to the delivery signal by the delivery sensor (which is indicative for the user touching the delivery surface).

In one embodiment, the at least one sensor or switch of which the response rate is changed or which is deactivated is the same sensor or switch generating the signal of the user proximity detection unit in response to which its response rate is changed or in response to which it is deactivated. This sensor may be the setting sensor, for example. In operation of the system, detecting the point of release of the setting surface may not be needed as the dose setting operation is followed by the dose delivery operation, which preferably is the one to be monitored by the motion sensing unit. Hence, the release of the setting surface may be characterized electronically by the delivery signal and/or the motion signals. The setting sensor may be reactivated after a timeout, e.g. when no delivery and/or no motion signal is generated within a predetermined time after the setting sensor had been deactivated.

In one embodiment, the at least one sensor or switch of which the response rate is changed or which is deactivated is different from the sensor or switch generating the signal of the user proximity detection unit in response to which the response rate is changed or the at least one sensor or switch is deactivated.

“Distal” is used herein to specify directions, ends or surfaces which are arranged or are to be arranged to face or point towards a dispensing end of the drug delivery device or components thereof and/or point away from, are to be arranged to face away from or face away from the proximal end. On the other hand, “proximal” is used to specify directions, ends or surfaces which are arranged or are to be arranged to face away from or point away from the dispensing end and/or from the distal end of the drug delivery device or components thereof. The distal end may be the end closest to the dispensing and/or furthest away from the proximal end and the proximal end may be the end furthest away from the dispensing end. A proximal surface may face away from the distal end and/or towards the proximal end. A distal surface may face towards the distal end and/or away from the proximal end. The dispensing end may be the needle end, where a needle unit is or is to be mounted to the device, for example.

In a particularly advantageous embodiment, an electronic system for a drug delivery device comprises:
at least one user interface member, which is arranged to be manipulated by a user of the drug delivery device, wherein the user interface member has an exterior operation surface arranged and configured to be touched by a user of the drug delivery device during operation of the drug delivery device, and wherein the electronic system further comprises
an electrical user proximity detection unit which is configured to generate a signal, when the user is close to or touches the exterior operation surface.

In another particularly advantageous embodiment, an electronic system for a drug delivery device comprises:
at least one user interface member, which is arranged to be manipulated by a user of the drug delivery device, wherein the user interface member has an exterior operation surface arranged and configured to be touched by a user of the drug delivery device during operation of the drug delivery device, and
an electrical connection detection unit, wherein the connection detection unit is configured to detect whether the electronic system is connected to a drug delivery device unit and wherein, when the connection to the drug delivery device unit is detected by the connection detection unit, the electronic system is configured to change its state, e.g. from non-operational to operational, e.g. in response to a connection signal generated or provided by the connection detection unit, the connection detection signal, preferably being indicative for the connection between the drug delivery device unit and the electronic system.

Features, which are disclosed in conjunction with different aspects and embodiments may be combined with one another even if such a combination is not explicitly discussed.

Further aspects, embodiments and advantages will become apparent from the following description of the exemplary embodiments in conjunction with the drawings.

### BRIEF DESCRIPTION OF THE FIGURES

FIG. 1 shows an embodiment of a drug delivery device.

FIG. 2 illustrates schematically an electronic system for a drug delivery device, e.g. the one in FIG. 1.

FIGS. 3A to 3F illustrate an embodiment of the electronic system.

FIGS. 4A and 4B illustrate an embodiment of the electronic system.

FIGS. 5A to 5C illustrate an embodiment of the electronic system.

FIGS. 6A to 6C illustrate an embodiment of the electronic system.

FIG. 7 illustrates an embodiment of the electronic system.

FIG. 8 illustrates another embodiment of the electronic system.

FIGS. 9A to 9D illustrate embodiments of method of operating an electronic system.

### DETAILED DESCRIPTION

In the figures, identical elements, identically acting elements or elements of the same kind may be provided with the same reference numerals.

In the following, some concepts will be described with reference to an insulin injection device. The systems described herein may be implemented in this device. The present disclosure is however not limited to such an application and may equally well be used for or in injection devices that are configured to eject other medicaments or drug delivery devices in general, preferably pen-type devices and/or injection devices.

In the following, embodiments are provided in relation to injection devices, in particular to variable dose injection devices, which record and/or track data on doses delivered thereby. These data may include the size of the selected dose and/or the size of the actually delivered dose, the time and date of administration, the duration of the administration and the like. Features described herein may include power management techniques (e.g. to facilitate small batteries and/or to enable efficient power usage).

Certain embodiments in this document are illustrated with respect to an injection device where an injection button and grip (dose setting member or dose setter) are combined e.g. similar to Sanofi's ALLSTAR® device. The injection button may provide the user interface member for initiating and/or performing a dose delivery operation of the drug delivery device. The grip or knob may provide the user interface member for initiating and/or performing a dose setting operation. The devices may be of the dial extension type, i.e. their length increases during dose setting. Other injection devices with the same kinematical behaviour of the dial extension and button during dose setting and dose expelling operational mode are known as, for example, the Kwikpen® or Savvio® device marketed by Eli Lilly and the FlexPen®, FlexTouch® or Novopen® device marketed by Novo Nordisk. An application of the general principles to these devices therefore appears straightforward and further explanations will be omitted. However, the general principles of the present disclosure are not limited to that kinematical behaviour. Certain other embodiments may be conceived for application to injection devices where there are separate injection button and grip components/dose setting members e.g. Sanofi's SoloSTAR®. Thus, the present disclosure also relates to systems with two separate user interface members, one for the dose setting operation and one for the dose delivery operation. In order to switch between a dose setting configuration of the device and a dose delivery configuration, the user interface member for dose delivery may be moved relative to the user interface member for dose setting. If one user interface member is provided, the user interface member may be moved distally relative to a housing. In the course of the respective movement, a clutch between two members of the dose setting and drive mechanism of the device changes its state, e.g. from engaged to released or vice versa. When the clutch, e.g. formed by sets of meshing teeth on the two members, is engaged, the two members may be rotationally locked to one another and when the clutch is disengaged or released, one of the members may be permitted to rotate relative to the other one of the two members. One of the members may be a drive member or drive sleeve which engages a piston rod of the dose setting and drive mechanism. The drive sleeve may be designed to rotate relative to the housing during dose setting and may be rotationally locked relative to the housing during dose delivery. The engagement between drive sleeve and piston rod may be a threaded engagement. Thus, as the drive sleeve cannot rotate during dose delivery, axial movement of the drive sleeve relative to the housing will cause the piston rod to rotate. This rotation may be converted into axial displacement of the piston rod during the delivery operation by a threaded coupling between piston rod and housing.

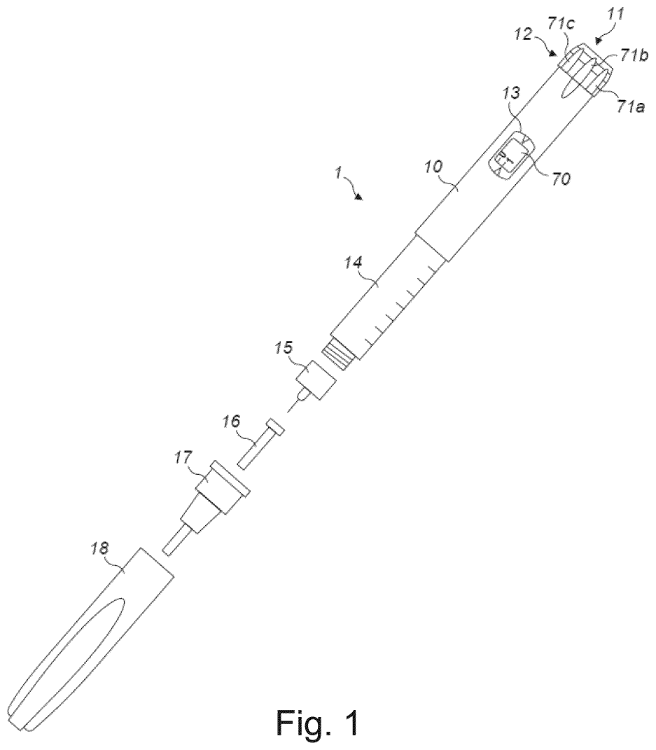

The injection device 1 of FIG. 1 is an injection pen that comprises a housing 10 and contains a container 14, e.g. an insulin container, or a receptacle for such a container. The container may contain a drug, e.g. insulin. The container may be a cartridge or a receptacle for a cartridge which may contain the cartridge or be configured to receive the cartridge. A needle 15 can be affixed to the container or the receptacle. The container may be a cartridge and the receptacle may be a cartridge holder. The needle is protected by an inner needle cap 16 and either an outer needle cap 17 or another cap 18. An insulin dose to be ejected from injection device 1 can be set, programmed, or ‘dialled in’ by turning a dosage knob 12, and a currently programmed or set dose is then displayed via dosage window 13, for instance in multiples of units. The units may be determined by the dose setting mechanism which may permit relative rotation of the knob 12 to the housing 10 only in whole-number multiples of one unit setting increment, which may define one dosage increment. This may be achieved by an appropriate ratchet system, for example. The indicia displayed in the window may be provided on a number sleeve or dial sleeve 70. For example, where the injection device 1 is configured to administer human insulin, the dosage may be displayed in so-called International Units (IU), wherein one IU is the biological equivalent of about 45.5 micrograms of pure crystalline insulin (1/22 mg). Other units may be employed in injection devices for delivering analogue insulin or other medicaments. It should be noted that the selected dose may equally well be displayed differently than as shown in the dosage window 13 in FIG. 1.

The dosage window 13 may be in the form of an aperture in the housing 10, which permits a user to view a limited portion of a dial sleeve 70 that is configured to move when the dosage knob 12 is turned, to provide a visual indication of a currently programmed dose. The dosage knob 12 is rotated on a helical path with respect to the housing 10 when turned during programming.

In this example, the dosage knob 12 includes one or more formations 71a, 71b, 71c to facilitate attachment of a data collection device or electronic system. An electronic system which may be attachable to the user interface member (knob 12 and/or button 11) or, in general, to elements or members of a dose setting and drive mechanism of the drug delivery device 1 will be described in more detail below. The electronic system may be provided within the user interface member, for example. The electronic system which will be described in more detail below can also be configured as an add-on for a drug delivery device.

The injection device 1 may be configured so that turning the dosage knob 12 causes a mechanical click sound to provide acoustical feedback to a user. In this embodiment, the dosage knob or dose button 12 also acts as an injection button 11. When needle 15 is stuck into a skin portion of a patient, and then dosage knob 12/injection button 11 is pushed in an axial direction, the insulin dose displayed in display window 13 will be ejected from injection device 1. When the needle 15 of injection device 1 remains for a certain time in the skin portion after the dosage knob 12 is pushed home, the dose is injected into the patient's body. Ejection of the insulin dose may also cause a mechanical click sound, which is however different from the sounds produced when rotating the dosage knob 12 during dialing of the dose.

In this embodiment, during delivery of the insulin dose, the dosage knob 12 is returned to its initial position in an axial movement, without rotation, while the dial sleeve 70 or number sleeve 70 is rotated to return to its initial position, e.g. to display a dose of zero units. As noted already, the disclosure is not restricted to insulin but should encompass all drugs in the drug container 14, especially liquid drugs or drug formulations.

Injection device 1 may be used for several injection processes until either the insulin container 14 is empty or the expiration date of the medicament in the injection device 1 (e.g. 28 days after the first use) is reached.

Furthermore, before using injection device 1 for the first time, it may be necessary to perform a so-called “prime shot” to ensure fluid is flowing correctly from insulin container 14 and needle 15, for instance by selecting two units of insulin and pressing dosage knob 12 while holding injection device 1 with the needle 15 upwards. For simplicity of presentation, in the following, it will be assumed that the ejected amounts substantially correspond to the injected doses, so that, for instance the amount of medicament ejected from the injection device 1 is equal to the dose received by the user.

As explained above, the dosage knob 12 also functions as an injection button 11 so that the same component is used for dialling/setting the dose and dispensing/delivering the dose. Again, we note that a configuration with two different user interface members which, preferably only in a limited fashion, are movable relative to one another is also possible. The following discussion will, however, focus on a single user interface member which provides dose setting and dose delivery functionality. In other words, a setting surface of the member which is touched by the user for the dose setting operation and a dose delivery surface which is touched by the user for the dose delivery operation are immovably connected. Alternatively, they may be movable relative to one another, in case different user interface members are used. During the respective operation, the user interface member is preferably moved relative to the body or housing of the device. During dose setting the user interface member is moved proximally and/or rotates relative to the housing. During dose delivery, the user interface member moves axially, e.g. distally, preferably without rotating relative to the housing or body.

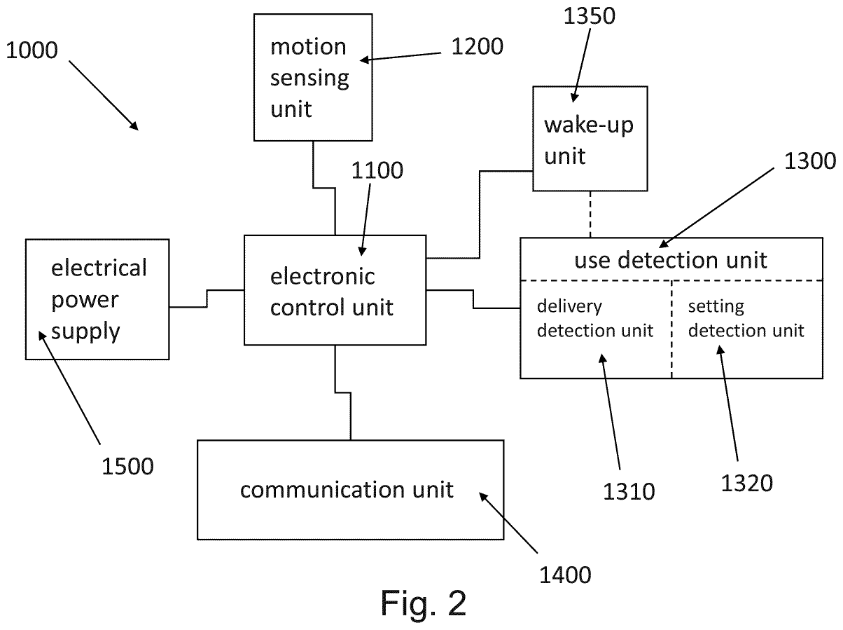

In the following, a general setup for an electronic system for a drug delivery device is disclosed. FIG. 2 illustrates a general configuration of elements of an electronic system 1000 which can be used in a drug delivery device, for example the device discussed further above or other devices.

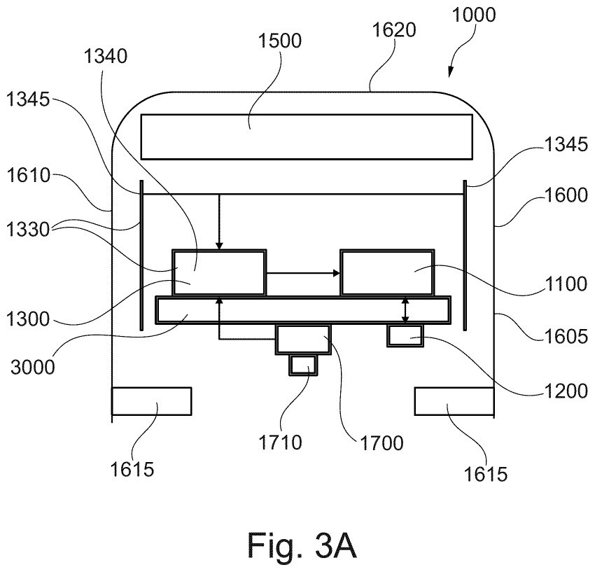

The electronic system 1000 comprises an electronic control unit 1100. The control unit may comprise a processor, e.g. a microcontroller or an ASIC. Also, the control unit 1100 may comprise one, or a plurality of memory units, such as a program memory and/or a main memory. The program memory may be designed to store program code which when carried out by the system controls operation of the system and/or the electronic control unit. The control unit 1100 is expediently designed to control operation of the electronic system 1000. The control unit 1100 may communicate via wired interfaces or wireless interfaces with further units of the electronic system 1000. It may transmit signals containing commands and/or data to the units and/or receive signals and/or data from the respective unit. The connections between the units and the electronic control unit are symbolized by the lines in FIG. 2. However, there also may be connections between the units, which are not illustrated explicitly. The control unit may be arranged on a conductor carrier, e.g. a (printed) circuit board (see 3000 in FIG. 3A). The other unit(s) of the electronic system may comprise one or more components which are arranged on the conductor carrier as well.

Electronic system 1000 further comprises an electrical motion sensing unit 1200. The motion sensing unit 1200 may comprise one sensor e.g. only one sensor, or a plurality of sensors. The motion sensing unit is expediently designed to generate motion signals, such as electrical signals, which are indicative for movement of one member of the electronic system or the drug delivery device relative to another member—e.g. movement of the dial sleeve or number sleeve relative to the drive sleeve or button/knob in the device discussed further above —, where the sensor may be fixedly connected to one of the members, e.g. the knob or button. The relative movement expediently occurs during the dose delivery operation. The respective sensor may be an optoelectronic sensor. The optoelectronic sensor may sense radiation emerging from a member moving relative to the sensor and impinging on the sensor to excite sensor signals or motion signals in the sensor, e.g. an optical encoder component. The radiation may be radiation reflected by the member and impinging on the member from a radiation source, such as an optoelectronic radiation source, e.g. an LED. The radiation source may be an IR source (IR-LED, an InfraRed Light Emitting Diode). The radiation source may be part of the sensor arrangement comprising the at least one sensor. One possible embodiment of the sensor is an IR-sensor which is configured to detect infrared light. The light source and the sensor may be arranged on the same component or member. The general functionality of optoelectronic sensor arrangements suitable for the electronic system discussed herein is disclosed in WO 2019/101962 A1, where the entire disclosure content is explicitly incorporated herein by reference for all purposes, especially as regards the different sensor arrangements and configurations. However, it should be noted that other sensor arrangement, e.g. using magnetic sensors, could be employed as well. In a motion sensing unit which has an electrically operated sensor and/or an electrically operated source for stimulating the sensor—such as a radiation emitter and an associated sensor—the power consumption may be particularly high and, hence, appropriate power management of electrical power available for powering the system may have a particular impact. The motion sensing unit 1200 may be designed to detect and preferably measure or quantify relative movement of one member of a dose setting and drive mechanism of or for the drug delivery device relative to another member of the dose setting and drive mechanism or relative to the housing 10 during a dose delivery operation. For example, the motion sensing unit may measure or detect relative rotational movement of two movable members of the dose setting and drive mechanism with respect to one another. Based on movement data received from or calculated from the signals of the unit 1200, the electronic system, e.g. the control unit, may calculate dose data, e.g. data on the currently delivered dose. The motion sensing unit 1200 is expediently configured to quantify the relative movement between a first member and a second member of the electronic system or the drug delivery device. The relative movement may be indicative for the delivered dose. The relative movement may be relative rotational movement. For example, the first member may rotate relative to the second member, such as during dose delivery. The motion sensing unit is expediently suitable to quantify the relative movement in whole-number multiples of one unit setting increment. The unit increment may be or may be defined by an angle greater than or equal to one of the following values: 5°, 10°. The unit setting increment may be or may be defined by an angle less than or equal to one of the following values: 25°, 20°. The unit setting increment may be between 5° and 25°, for example. The unit setting increment may correspond to a relative rotation of 15°, for example. The unit setting increment may be the rotation required to set the smallest settable dose to be delivered by the device. As has been explained above, the amount or distance of the relative movement determined by the motion sensing unit between the first and second members is characteristic for the currently set dose in a dose setting operation or for the currently dispensed dose in a dose delivery operation. The size of the dose delivered may be determined by or correspond to the distance by which a piston rod of the dose setting and drive mechanism is displaced distally relative to the housing during the dose delivery operation.

The electronic system 1000 further comprises a use detection unit 1300. The use detection unit may be associated with the user interface member or members (knob 12 or button 11 in the device discussed above) such that intended manipulation of or the manipulation of the member for setting and/or for delivering a dose may be detected. When the (intended) manipulation is detected, the use detection unit is configured to generate or triggers generation of a use signal. The use signal can be transmitted to the electronic control unit 1100. The electronic control unit 1100 may, in response to the signal, issue a command or signal to one of, an arbitrarily selected plurality of, or all of the other electrically operated units of the system, e.g. an activation or switching signal. For example, the control unit may cause the respective unit to be switched from a first state, e.g. a sleeping state or idle state with a lower power consumption or an off state with no power consumption, to a second state with an increased power consumption. The switching may be effected by an according switching command or signal issued by the electronic control unit to the respective unit. In response to the use signal all units may be switched to the second state or just selected units. If only selected units are switched to the second state with higher power consumption, it is expedient that these units are intended to be used during the operation, which is intended to be commenced by the user. One unit to be switched to the second state may be the motion sensing unit 1300. Another unit may be the communication unit, which is described in more detail below. For example, typical times required to switch a motion sensing unit to the second state are between 2 ms and 7 ms, such as between 2.5 ms and 3.2 ms, e.g. after generation of the use signal or actuation of the user interface member to initiate the operation of the system, e.g. the dose delivery operation.

The use detection unit 1300 can be selective with respect to the type of operation which is detected. That is to say, the use detection unit may be configured to distinguish between different operations which can be performed, e.g. between the dose setting operation and the dose delivery operation.

In one embodiment, the use detection unit comprises a delivery detection unit 1310 and/or a setting detection unit 1320. The setting detection unit is expediently configured to detect a dose setting operation. The deliver detection unit is expediently configured to detect a dose delivery operation. The system may comprise just one of the setting detection unit and the delivery detection unit, e.g. the setting detection unit or the delivery detection unit, or both of these units. The setting detection unit expediently provides a setting signal to the electronic control unit. The delivery detection unit may provide a delivery signal to the electronic control unit. The setting signal may be the use signal in response to which one or more further units are switched to a state of higher power consumption. Alternatively, the delivery signal may be the use signal in response to which one or more further units are switched to a state of higher power consumption. It is also possible that the setting signal is a first use signal, e.g. causing activation of the motion sensing unit, and that the delivery signal is a second use signal, e.g. used to activate the communication unit, e.g. in order to transmit the dose information derived from the motion sensing unit to another device.

The use detection unit 1300 comprises a user proximity detection unit (not explicitly shown in this representation), which is discussed later on in more detail. The user proximity detection unit is configured to generate a signal, e.g. the use signal or a proximity signal, when the user is close to or touches the exterior operation surface. For the setting detection unit, proximity relative to a setting surface as exterior operation surface of the user interface member may be detected. For the delivery detection unit, proximity relative to a delivery surface as exterior operation surface of the user interface member may be detected.

In alternative embodiments, the system may not comprise a use detection unit but just a user proximity detection unit or a user proximity detection unit which operates independently of the use detection unit, either entirely or only for some operations. The user proximity detection unit is expediently configured to generate a proximity signal when the user is close to or touches the respective surface of the user interface member which is monitored by the proximity detection unit. This signal may be the use signal, for example, but may be used for other purposes as well. The user proximity detection unit may be configure to detect proximity relative to the setting surface of the user interface member and/or relative to the delivery surface. Separate user proximity detection units may be provided for the setting surface and the delivery surface or a common user proximity detection unit may be provided for both surfaces.

The use detection unit 1300, when it is operational to generate the use signal, e.g. the setting signal and/or the delivery signal, expediently has a lower power consumption than the motion sensing unit 1200 when the motion sensing unit 1200 is active. The same holds for the user proximity detection unit. As noted already, referrals to the use detection unit should be understood as referrals to the user proximity detection and vice versa, unless otherwise indicated in the context.

The electronic system 1000 further comprises a communication unit 1400, e.g. an RF, WiFi and/or Bluetooth unit, such as Bluetooth low energy (BLE). The communication unit may be provided as a communication interface between the system or the drug delivery device and an external device, such as other electronic devices, e.g. mobile phones, personal computers, laptops and so on. For example, dose data may be transmitted by the communication unit to the external device and/or synchronized with the device. The dose data may be used for a dose log or dose history established in the external device. The communication unit may be provided for wireless communication.

Electronic system 1000 further comprises an electrical power supply 1500, such as a rechargeable or non-rechargeable battery. The power supply 1500 may provide electrical power to the respective units of the electronic system.

In one embodiment, the power consumption, in particular the maximum power consumption, of the electronic system in the first state, e.g. prior to generation of the use signal, may be less than or equal to one of the following values: 300 nA, 250 nA, 200 nA (nA: nanoampere). Alternatively or additionally, in the second state of the electronic system, the power consumption, in particular the minimum power consumption, may be greater than or equal to one of the following values: 0.5 mA, 0.6 mA, 0.8 mA (mA: milliampere). The difference may result from the power consumption of the motion sensing unit and/or of the communication unit which may be active or operable in the second state and switched off or in a sleep state in the first state of the electronic system.

In one embodiment, the power consumption P2 in the second state may be greater than or equal to at least one of the following values: 2*P1, 3*P1, 4*P1, 5*P1, 10*P1, 20*P1, 30*P1, 40*P1, 50*P1, 100*P1, 500*P1, 1000*P1, 2000*P1, 5000*P1, 10000*P1 where P1 is the power consumption in the first state. In the second state, the motion sensing unit may be active and/or the communication unit may be active, e.g. for wireless communication.

When the system is in the first state, e.g. with neither the motion sensing unit being active nor the communication unit, the current consumption may be 200 nA. When (only) the motion sensing unit is active, the power consumption may be 0.85 mA. When the communication unit is active, e.g. in addition to the motion sensing unit or only the communication unit, the power consumption may be 1.85 mA.

Although not explicitly depicted, the electronic system may comprise a, preferably permanent and/or non-volatile, storage or memory unit, which may store data related to the operation of the drug delivery device such as dose (history) data, for example.

In one embodiment, the electronic control unit 1100 is configured to reduce the power consumption of the respective unit, i.e. to switch the unit back to the first state. This is suitable, for example, if an event which is relevant for that unit, e.g. a motion sensing event (motion signal) for the motion sensing unit, has not occurred in a predetermined time interval after the unit has been switched from the first state into the second state and/or after the use signal has been generated. The monitoring of the time interval may be achieved by a timer unit which is operatively connected to the electronic control unit (not explicitly shown). In case, after the use signal, there is no signal generated by the motion sensing unit within the predetermined time interval, the entire system may be switched to the first state again. This time interval may be greater than or equal to one of the following values 5 s, 10 s, 15 s, 20 s, 25 s, 30 s. Alternatively or additionally the time interval may be less than or equal to one of the following values: 50 s, s, 40 s, 35 s, 30 s. The time interval may be between 5 and 50 seconds, e.g. 30 s. The entire system may be switched back to the first state in case no motion signal is generated within the predetermined time interval. The predetermined time interval is expediently constant.

Alternatively or additionally, the electronic system may be configured to switch back to the first state, in case the user proximity detection unit does not detect the proximity of a user to the respective surface for a predetermined time interval, e.g. by not generating a proximity signal, after the system has been switched to the second state. The time interval may be chosen as discussed in the preceding paragraph.

However, in an alternative embodiment, the user proximity detection unit may also be switched off after the use signal has been generated or the control unit may be configured to ignore signals of the user proximity detection unit, especially the one used for generating the use signal.

The electronic system 1000 further comprises a wake-up unit 1350. Wake up unit 1350 may be configured to generate a wake-up signal, e.g. an electrical signal, which causes the use detection unit 1300 and/or the user proximity detection unit such as one or more sensors or switches thereof to be switched to a state of higher power consumption. The wake-up unit may cause an increase of a non-zero power consumption or switch the power on for the respective sensor or switch. Consequently, when the unit is in the state of lower power consumption, generation of the use signal or proximity signal may not be possible without having generated a wake-up signal previously. The wake-up signal may be generated by the wake-up unit. The wake-up signal may either be transmitted to the electronic control unit, which then causes the use detection unit 1300 to become operational, e.g. by an according activation signal, or, alternatively, by way of a direct connection between the wake-up unit and the use detection unit (illustrated by the dashed line in FIG. 2), transmitted directly to the use detection unit in order to switch the use detection unit to the state of higher power consumption. The use detection unit may comprise the delivery detection unit 1310 and/or the setting detection unit 1320. The wake-up unit may cause switching of the user proximity detection unit, e.g. one of the setting detection unit or one of the delivery detection unit, to a state of higher power consumption. In an embodiment, alternatively or additionally to the wake-up unit, the setting detection unit may be used to wake-up the delivery detection unit.

The event which triggers the wake-up signal may be an event which is expected to be likely followed by a dose setting operation and/or a dose delivery operation, i.e. an operation which should be causal for issuance of the use signal in order to power-up other electronic units of the electronic system such as the motion sensing unit 1200 and/or the communication unit 1400, for example. Events which could be used to trigger the wake-up signal may be:
vibrations which are detectable by a vibration sensor of the wake-up unit. Typically, if a drug delivery device is removed from the item in which it is carried, such as a bag, this is done in order to use the device. Therefore, in case vibrations are detected, this could be used as an indicator of an upcoming dose setting or dose delivery operation. The vibration sensor expediently detects the vibration independent of the orientation of the system or the device.
changes in orientation which are detectable by an orientation sensor, such as comprising a gyroscopic sensor (accelerometer), or another sensor which generates a signal once a predetermined orientation is assumed e.g. relative to the center of the earth gravity. Accelerometers usually have a higher power consumption in the active state than vibration sensors, which is why vibration sensors may be preferred.

In case the use signal is not generated within a predetermined time after the use detection unit has been switched to the state of higher power consumption, the system may be configured, such that the use detection unit can, again, be switched to a state of lower power consumption as then, it can be assumed that the device will not be operated anytime soon. This predetermined time may be less than or equal to any of the following values: 10 s, 9 s, 8 s, 7 s, 6 s, 5 s, 4 s, 3 s. This may be achieved by an appropriate timer circuit, for example. From that state, the use detection unit may be woken again by the wake-up unit.

The wake-up signal may be generated before the dose setting operation is being commenced or the setting surface of the user interface member is touched and/or before the user is even close to that surface. Thus, the wake-up signal may be used reliably to power-up the use detection unit such that it may perform its function.

Expediently, the power consumption of the wake-up unit is smaller than the power consumption of the use and/or user proximity detection unit when it is operational, i.e. in the state of higher power consumption.

The respective unit which has been described above may be integrated into a user interface member of the electronic system which is discussed in further details below in conjunction with various embodiments.

It goes without saying that the electronic system 1000 may comprise further electronic units other than the ones shown such as other sensing units, which sense or detect different quantities or events than the relative movements which the motion sensing unit detects.

In the following some embodiments of the use detection unit and/or the user proximity detection unit are described.

FIG. 3A schematically illustrates an embodiment of an electronic system 1000. The system 1000 comprises a user interface member 1600. The user interface member is designed to be operated during a dose setting operation and/or a dose delivery operation by the user. The user interface member 1600 has different exterior operation surfaces. The operation surfaces may be defined by exterior surfaces which are accessible from the exterior of a user interface member body or housing 1605. The user interface member 1600 has a setting surface 1610 which is arranged to be gripped by the user for dose setting, e.g. with two fingers such as the index finger and the thumb. The setting surface is a radially facing surface which circumferentially delimits the user interface member 1600. The user interface member 1600 also has a delivery surface 1620. The delivery surface is arranged to be contacted, e.g. pressed, by the user for dose delivery. The delivery surface 1620 is an axially oriented surface, e.g. a proximally facing surface. As noted above, embodiments of the disclosure can employ different user interface members for setting and delivery.

Within the user interface member 1600, e.g. within an interior hollow defined by the user interface member body 1605, some additional elements or units of the electronic system are housed. Specifically, the electronic system comprises the electronic control unit 1100. The system also comprises a conductor carrier 3000, e.g. a circuit board such as a printed circuit board. Conductors on the conductor carrier may conductively connect the electronic control unit to further electrical or electronic units or members of the system. The electronic control unit is arranged on the conductor carrier, e.g. mounted to the carrier.

The electronic system comprises a user proximity detection unit 1330, in particular as a use detection unit for generating the use signal. In this embodiment, the user proximity detection unit is configured to detect whether the user's finger is close to the setting surface 1610. The user proximity detection unit 1330 comprises a sensor 1340, e.g. a capacitive sensor. The sensing part of the sensor 1340, e.g. integrated into a chip on the conductor carrier 3000, is operatively, e.g. conductively, connected with a sensor electrode 1345 of the sensor or of the user proximity detection unit. The capacitive sensor may be a low-power controller that monitors input capacitance from the sensor electrode 1345 and outputs a signal used to wake the electronic control unit and/or the motion sensing unit if the capacitance detected has exceeded a predetermined value, e.g. a value typical for the human skin. The sensor can be configured to output the signal when the user is less than 0.5 mm away from the monitored surface. We note, however, that the operating distance of the sensor can also be increased, if desired. Of course, it should be ensured that a user touching the surface which is monitored by the capacitive sensor always generates a relevant or significant signal. The operating distance can be tuned by the design of the electrode, and by the selection of appropriate sensor (signal) thresholds, e.g. in the software.

The sensor electrode 1345 has electrode portions which are assigned to the surface which should be monitored by the detection unit for the user being proximate to or touching the surface. In the presently depicted embodiment, the sensor electrode portions face towards the setting surface 1610. The conductive connection of the electrode and the sensor 1340 is symbolized by the line between the electrode 1345 and the sensor 1340. Electrode portions which are angularly adjacent to one another may be separated by a free space. That is to say not the entire surface to be monitored by the sensor has to be covered by the electrode. The user interface member body 1605 is arranged between the electrode 1345 and the exterior surface such that the user does not touch the electrode 1345 when touching the exterior surface in the region overlapping with the electrode 1345. It is, however, also conceivable that the user can touch the electrode. Having the electrode in the interior, however, facilitates sealing the interior against external influences. The electrode 1345 may provide a large surface area of sensitivity for the capacitive sensor. The electrode may be a pressed metal component, for example. The sensor 1340 is arranged on and preferably mounted on carrier 3000. The sensor may be or may comprise a (low-power) controller which monitors input capacitance from the sensor electrode 1345. The sensor 1340 is expediently configured to perform a sensor measurement or acquire a sensor signal with a frequency (polling frequency) or response rate of less than or equal to one of the following values: 10 Hz, 5 Hz, 4 Hz, 3 Hz, 2 Hz, 1 Hz. The power consumption of the sensor 1340 may be less than or equal to one of the following values: 15 μA, 10 μA, 8 μA, 7 μA, 6 μA, 5 μA, 4 μA, 3 μA. It will be appreciated that the power consumption depends on the frequency or rate with which measurements are taken. The more often the sensor is polled, i.e. a measurement is conducted, the higher the power consumption will be. The order of magnitude of the power consumptions mentioned above may apply for the frequencies stated above. The frequencies are particularly suitable, if the sensor 1340 monitors proximity to the setting surface of the user interface member. Since the time between dose setting and dose delivery is comparatively high as the user needs to switch between the two surfaces for operating the system or the device, such small frequencies are sufficient. In case the delivery surface is monitored by the sensor, the frequency may have to be greater than or equal to one of the following values: 40 Hz, 50 Hz, 60 Hz, 70 Hz, 80 Hz, 90 Hz, 100 Hz. This involves a higher power consumption, e.g. greater than or equal to one of the following values: pA, 40 μA, 50 μA, 60 μA, 70 μA, 80 μA, 90 μA, 100 μA. The sensor 1340 and the control unit 1100 may be mounted on the same surface of the carrier 3000.

The system furthermore comprises the motion sensing unit 1200 which is only schematically symbolized and, preferably comprises one or more optoelectronic sensors and/one or more associated radiation emitters, e.g. IR sensors and IR emitters. The motion sensing unit may be bidirectionally conductively connected to the electronic control unit 1100 as hinted by the double arrow. One direction may be the one where the activation signal is transmitted from the electronic control unit to the motion sensing unit. In the other direction, motion signals may be sent from the motion sensing unit to the control unit, which may process the signal furthers, e.g. to calculate dose information. The motion sensing unit 1200 may be arranged on that side of the conductor carrier 3000 which faces away from the control unit 1100.

Also, the system 1000 comprises the power supply 1500, e.g. a battery, such as a coin cell. The power supply may be configured to provide a total charge of approx. 50-500 mAh at a voltage of approx. 1.4-3V. This may be achieved or assisted by stacking multiple coin cells, for example. The power supply 1500 is conductively connected or connectable to the other components of the electronic system, which require electrical power for operating. The conductive connection is not explicitly illustrated in FIG. 3A. The power supply may, however, be arranged so as to extend along one main surface of the conductor carrier 3000 as depicted. The power supply, in the depicted embodiment, is arranged between the conductor carrier 3000 and the delivery surface 1620. This facilitates a compact formation of the user interface member.

A radial width or diameter of the user interface member as seen from the exterior of the member, e.g. in top view onto the delivery surface, may be less than or equal to one of the following values: 2 cm, 1.5 cm. Alternatively or additionally, the radial width or diameter of the user interface member may be greater than or equal to one of the following values: 0.5 cm, 0.7 cm. The radial extension may be determined relative to the rotation axis of the user interface member during dose setting or relative to the main longitudinal axis of the user interface member, which axes may coincide. The length or axial extension of the user interface member 1600 may be less than or equal to one of the following values: 2 cm, 1.5 cm. Alternatively or additionally, the length or axial extension of the user interface member 1600 may be greater than or equal to one of the following values: 0.5 cm, 0.7 cm. For example, the radial width of the user interface member can be 18 mm and the length can be 19 mm.

The sensor is configured to output a sensor or use signal which is used to switch or to trigger switching of the electronic control unit 1100 to a state of higher power consumption and/or causes the electronic control unit to activate the motion sensing unit 1300. For example, if the sensed capacitance exceeds a predetermined threshold, the use signal may be issued.

Electronic system 1000 is configured to be connected to, preferably configured to be releasably connected to, a drug delivery device unit as an add-on unit or module. The drug delivery device unit may be electronic free. Accordingly, all electronics may be provided in the electronic system. The drug delivery device unit may be disposable. That is to say, the unit can be disposed of after a reservoir of the unit has been emptied using the drug delivery device comprising the unit and the system 1000. The electronic system 1000 could be reused for another drug delivery device unit. The drug delivery device unit is preferably configured as fully functional on its own, i.e. it could be operated for setting a dose to be delivered and deliver the set dose. One exemplary unit is the one depicted in FIG. 1. The electronic system may be a pure add-on to an, otherwise, fully functional unit. Alternatively, a drug delivery device may comprise the electronic system as an integral part, i.e. a part which is either disposed of together with the remainder of the device or necessary such that the device can be operated for setting and delivering a dose of drug, e.g. because without the electronic system the drug delivery device unit would lack a surface accessible for the user for conducting a dose setting operation or a dose delivery operation. For a connection to the drug delivery device unit, the electronic system may comprise one or more connection features 1615, e.g. snap features. The respective connection feature is arranged in a distal portion of the user interface member 1600, e.g. in the interior of the member. The system 1000 is expediently configured to be mechanically connected, either permanently or removably (in other words: in a removable way), to a member of the drug delivery device unit such as a member of the dose setting and drive mechanism, e.g. to a drive sleeve or the dose knob and/or the injection button of the unit discussed in conjunction with FIG. 1. The system, e.g. via the user interface member body 1605, may be rotationally and axially locked to the member of the drug delivery device unit. The member to which the system is connected may be movable relative to the housing 10 during dose setting and dose delivery, e.g. rotationally and/or axially during setting and, e.g. only, axially during delivery. The member can engage the piston rod threadedly, for example. The dose knob and the drive sleeve of the unit in FIG. 1 may be formed integral or act as a single member during dose setting and dose delivery. During dose setting, the drive sleeve may be selectively rotationally locked to a dial sleeve of the dose setting and drive mechanism such that the dial sleeve and the drive sleeve co-rotate during dose setting, e.g. by a clutch, and the dial sleeve rotates relative to the drive sleeve during dose delivery. The dial sleeve may be the number sleeve or be coupled to the number sleeve. The relative rotation between dial sleeve and drive sleeve during dose delivery may be measured by the motion sensing unit. However, it will be readily apparent to those skilled in the art that the disclosed concepts will also work with dose setting and drive mechanisms having different functionalities.

It should be readily apparent for one of skill in the art that instead of having a setting sensor as in the depicted embodiment, a delivery sensor which monitors proximity to or touching of the delivery surface 1620 could be applied as an alternative or in addition to the setting sensor. The delivery sensor may also be or comprise a capacitive sensor.

The electronic system 1000 as depicted in FIG. 3A comprises a connection detection unit 1700. Such a unit may be employed in any of the systems disclosed herein. The connection detection unit 1700 may be configured to detect whether the electronic system is connected to a drug delivery device unit. The system may be configured, such that, in case a connection to the drug delivery device unit is detected, the electronic system is switched to a state of higher power consumption. In case the connection detection unit detects a connection to a drug delivery device unit, a detection signal may be generated or triggered. The electronic system may be configured such that, in response to the detection signal, the electronic system 1000 is switched to the state of higher power consumption.

For example, the connection detection unit 1700 may cause the power supply 1500 to be conductively connected to other components or units of the electronic system, thereby enabling these components or units to function, when appropriately activated, e.g. by the electronic control unit. Specifically, when connection to the drug delivery device unit is detected, power from the power supply could be provided to the respective component or units. When no connection is detected, the power supply may be disconnected from the respective component or unit of the electronic system. Hence, the connection detection unit may be or may be connected to an interrupter which shuts off the power supply unless the connection to the device unit is detected.

Alternatively or additionally, the connection detection unit 1700 is configured to switch the use detection unit 1300 or the user proximity detection unit, e.g. only the respective detection unit, to a state of higher power consumption, e.g. a state where the proximity or use detection unit could be operated. Before being switched to the state of higher power consumption by the connection detection unit, the proximity or use detection unit may be non-operable in a first state.

Hence, the connection detection unit 1700 may be connected to the electronic control unit and cause the respective units to be activated or switch to the state of higher power consumption via the electronic control unit or it may be connected directly to the respective unit which should be switched in to the state of higher power consumption by the connection detection unit. The latter situation is depicted in FIG. 3A, where the connection detection unit is connected directly to the use detection unit 1300 and/or user proximity detection unit 1330. The connection detection unit 1700 may comprise a switch 1710, such as a micro (force) switch, for example. The switch may be arranged on a side of the carrier 3000 facing towards an opening of the user interface member body 1605. The opening may be designed to receive the member of the device unit when the device unit and the system are connected. The switch 1710 may be arranged to mechanically contact the member of the drug delivery device unit and/or arranged to be triggered by the member. When the switch is triggered, the use detection unit or user proximity detection unit may be rendered operable. In this way, power drain of the unit during storage may be avoided or at least reduced substantially.

For example, when the system comprises a capacitive sensor as has been discussed above for the user proximity detection unit, it may be necessary to conduct measurements periodically, e.g. with a frequency or response rate as mentioned above, when the sensor operates to check whether a user is close to the relevant surface. Every measurement draws electrical power from the power supply. Thus, when the system is not connected to a drug delivery device unit, such a power drain would be completely unnecessary. Hence, the connection detection unit improves power management, especially during terms of storage of the electronic system when separate from a drug delivery device unit.

We note that a connection detection unit 1700 as discussed above is not only suitable for the use detection unit or user proximity detection unit but also for other units of an electronic system such as the electronic control unit, the motion sensing unit, the communication unit or even other units. However, in case a use detection unit 1300 is applied to trigger the switching of the system to a state of higher power consumption, e.g. a state where the motion sensing unit and/or the communication unit is active, the connection detection unit which controls the state of the use detection unit is particularly advantageous. Also, it is not necessary that the use detection unit comprises a capacitive sensor. Rather, other sensors may also be suitable for being operated together with the connection detection unit to be switched to a state of higher power consumption, e.g. to an active state from an inactive state. For example, the connection detection unit may also operate with a setting sensor, a delivery sensor and/or a wake-up unit whatever is feasible in the specific situation.

When the connection detection unit does not detect that the system is connected to a device unit, the power consumption may be zero or smaller than the power consumption when only the use detection unit of the electronic system is active. When the use detection unit is not active, the electronic control unit may be either switched off or have a lower power consumption than when it is operative, e.g. to control the communication unit and/or the motion sensing unit.

It should be noted that the connection detection unit 1700 can be implemented in any of the embodiments disclosed herein or form an embodiment on its own.

FIGS. 3B to 3F illustrate one embodiment of the electronic system 1000, which in some aspects is more specific than the one discussed in conjunction with FIG. 3A. However, all features disclosed in conjunction with FIG. 3A should also apply for this embodiment and vice versa.

FIG. 3B shows a schematic sectional view of the electronic system 1000. The system, again, comprises the user interface member 1600 with user interface member body 1605 and the setting surface 1610 as well as the delivery surface 1620. The conductor carrier 3000 is provided in the interior of the user interface member body 1605 and carries one or more electronic units as described above. The conductor carrier is axially and rotationally secured in the user interface member. In the sectional view of FIG. 3B, not all of the units discussed previously are explicitly illustrated but may be present nevertheless.

The arrangement of the power supply 1500 and its contact connection to electrically powered components are illustrated in more detail than in the schematic representation of FIG. 3A. The power supply 1500 is arranged between the carrier 3000 and the proximal surface of the user interface member, i.e. the delivery surface 1620. A spacer component 1510 is arranged between the carrier 3000 and the power supply such that the power supply 1500 is supported on the carrier via the spacer component 1510. The spacer component 1510 may have a curved interior surface in order to extend circumferentially along the (electronic) components mounted on the carrier 3000. Further, FIG. 3B shows the power supply electrode 1520. The electrode 1520 is conductively connected to the power supply and to one or more electronic components, particularly via the conductor carrier 3000. The electrode 1520 has a contact portion 1530 which conductively connects the electrode to the conductor carrier 3000. The electrode 1520 extends from that side of the carrier 3000 which faces the power supply 1502 that side of the carrier 3000 which faces away from the power supply 1500. On the latter side, the contact portion 1530 is arranged. However, different configurations of the contact connection between power supply and electronic component(s) can also be envisaged. The electrode 1520 has one or more biasing portions 1540. The biasing portions 1540 are elastically deflectable. The respective biasing portion 1540 may be a flexible arm. The respective biasing portion may be provided to exert a force on the power supply 1500 such that the power supply is biased towards the carrier 3000 and/or the spacer, e.g. distally. The respective biasing portion may interact with an end face delimiting the interior of the user interface member proximally so as to bias the power supply 1500 towards the carrier 3000 via the electrode 1520. The biasing portions 1540 are connected by a ring portion 1560 and extend axially away from and inwardly relative to the ring portion 1560 (see FIG. 3C). A power supply contact portion 1570 which is conductively connected to the power supply extends from the ring portion inwardly (see FIG. 3C). The electrode 1520 has a securing portion 1550, e.g. diametrically opposite from the contact portion 1530. The securing portion 1550 may be designed to secure the power supply 1500 to the spacer component 1510. In the depicted embodiment, this is achieved by clamping the spacer component and the power supply between regions of the electrode.

The system 1000, in addition to the user interface member body 1605, has a chassis 1670. The chassis 1670 is, preferably rotationally and axially, locked to the user interface member body 1605, e.g. snap fitted or welded. The chassis together with the user interface member body may define an interior of the user interface member. The interior is preferably sealed, e.g. dust-tight and/or fluid-tight. For this purpose, one or more seals may be provided at the interface user interface member body/chassis (not explicitly illustrated). The chassis 1670 may close a distal opening of the user interface member body 1605. The chassis may comprise a rigid portion 1672 and/or a, preferably elastically, deformable portion 1674. The deformable portion may be laterally surrounded by the rigid portion, e.g. on a common surface.

The chassis defines the receiving space of the user interface member for receiving the member of the drug delivery device unit to which the system 1000 should be connected. The deformable portion 1674 of the chassis is preferably designed to interact with a member of the drug delivery device unit and, when the unit has been connected to the system, the deformable portion 1674 is preferably elastically deformed as compared to the situation when the system is not connected to the device unit. The movement during the deformation of the deformable portion 1674 may be used to trigger the switch 1710 of the connection detection unit 1700. When the system is disconnected from the device unit, the portion may resume its undeformed shape due to the elasticity, thereby causing the connection detection unit to detect a disconnection from the device unit, e.g. by the switch changing its state.

As the portion 1674 is part of the chassis 1670, a direct contact between the member of the drug delivery device unit and the switch 1710 is not required which facilitates provision of a sealed interior of the user interface member. The chassis 1670 may also comprise one or more light guide portions 1676. The light guide portion(s) may be operatively coupled to the radiation emitter and radiation sensor provided in the motion sensing unit, in case radiation is used for motion sensing. Consequently, movement of a member which moves relative to the end surface of the light guide 1676 remote from the interior of the user interface member 1600, e.g. an encoder component, may cause variations in the radiation (intensity) reflected back to the radiation sensor, where this radiation may be fed to the sensor through the light guide portion and/or the radiation preferably was generated by the radiation emitter. In this way, the movement of the member, e.g. the dial or number sleeve, relative to the user interface member 1600 or the dose knob or button may be quantified, such as during the dose delivery operation. The light guide portions and/or the according sensors may be out of phase relative to reflective portions of the encoder component (see WO 2019/101962 A1) which has some advantages.

The sensor electrode 1345, which may be formed from a metal, e.g. as a metal pressing, has circumferentially distributed and radially facing portions 1347 which are arranged to sense proximity or touch of the setting surface 1610 as discussed previously where only one of these portions is shown in FIG. 3B. The one or more portions of the sensor electrode may be arranged between an interior surface of the user interface member body 1605 and an exterior surface of the chassis 1670. Further, one or more portions of the electrode 1345 may be arranged between the chassis and the carrier 3000.

FIG. 3C shows an exploded view of the elements of the system depicted in FIG. 3B. The chassis 1670 is shown in more detail and a perspective view in FIG. 3D along with the electrode 1345 which provides touch sensitivity to the capacitive sensor on the carrier 3000. The electrode 1345 has a plurality of axially extending electrode portions 1347, e.g. four portions in the depicted embodiment. The chassis 1670 has a surface structure on its radial surface which is designed to receive one or more of the portions 1347 such that, by the axially extending portions, a rotational orientation between the electrode 1345 and the chassis 1670 may be defined. This has advantages during manufacturing. The electrode portions 1347 are connected with each other via a ring-like portion 1348, where the ring as in the depicted electrode may be open with two ends which face one another in the angular direction. Electrode 1345 further has a connection portion 1349 for a conductive connection of the electrode to the capacitive sensor 1340, see further above. This portion may be arranged between the chassis 1670 and the conductor carrier 3000.

FIG. 3E shows the situation where the electronic system 1000 as described previously has been connected to a drug delivery device unit. FIG. 3E shows a schematic sectional view of the situation when the system 1000 is mounted to the drug delivery device unit. In FIG. 3E, the user interface member 1600 as has been discussed previously is shown. In addition to the components of the electronic system 1000, a member 4000 of a dose setting and drive mechanism of the drug delivery device unit is shown. The member may be the dosage knob 12 or the injection button 11 of the device discussed further above, which could be integrated into one component. The member 4000, preferably, is a user interface member of the drug delivery device unit, in case this unit is fully operational without the electronic system. Alternatively, the member 4000 can be an interior member which is not accessible from the exterior but only provided for connection to the electronic system 1000. This is particularly suitable for electronic systems which are required for enabling a dose setting operation and/or dose delivery operation for a unit which, otherwise, would not function. The member 4000 has an interior 4030. Within this interior, a piston rod (not explicitly shown) of the dose setting and drive mechanism may be arranged. The piston rod may be arranged to drive the bung in a cartridge of the drug delivery device unit to dispense drug from the cartridge.

As illustrated in FIG. 3E, the electronic system 1000 is connected to the member 4000, preferably releasably, such that the electronic system can be reused for another drug delivery device unit or on a case-by-case basis with one drug delivery device unit such that the electronic system is fitted to that unit, e.g. before the setting operation for dispensing the desired dose is commenced, removed again, e.g. after the delivery operation for delivering the dose has been completed, and reconnected for the subsequent dose setting operation. In FIG. 3E, the connection is highlighted with reference numeral 4010 and can be a form-fitting connection such as a snap fit connection, for example. In the disclosed embodiment a flexible snap arm 1678 is provided on the chassis 1670, which may be axially oriented into the distal direction and/or engages a corresponding recess 4020 in the member 4000. It is readily appreciated that the specific type of connection used for connecting the electronic system to the drug delivery device unit can be varied, of course. It is advantageous if the electronic system is rotationally and axially locked to the drug delivery device unit or the member 4000 as tightly as possible, as the electronic system 1000 comprises the motion sensing unit and any relative movement allowed by the connection between the electronic system and the drug delivery device unit might influence the data quality of data retrieved from the signals of the motion sensing unit, as the relative movement would contribute considerably to the measuring errors. As shown in FIG. 3E, a mechanical contact portion 4040 of member 4000, e.g. a protrusion, causes actuation of the switch 1710 and, hence, causes the connection detection unit to switch the electronic system to a state of higher power consumption, e.g. by switching on the use detection unit or the user proximity detection unit (not explicitly shown in this figure). Portion 4040 may contact the deformable portion 1674 of the chassis which then may transfer the force from portion 4040 to the switch 1710 in order to cause generation of the according signal.

FIG. 3F shows the top view onto a proximal end surface of the member 4000. The contact portion 4040 is provided in the central region 4050 of the member 4000. This region is separated from an edge region 4060, e.g. by a groove 4070. The contact portion 4040 may be elevated with respect to the surrounding surface of the central portion 4050. The (radial) recesses 4020 for engaging the snap arms 1678 may be connected to the groove 4070 and/or protrude radially from the groove 4070. Preferably, the recesses 4020 are radially outwardly oriented. Member 4000 further has one or more axial openings 4080. The respective opening may penetrate through the entire body of the member 4000. The respective opening 4080 may be provided to establish optical communication between the motion sensing unit, e.g. via the light guide portion(s) 1676, and an other member of the dose setting and drive mechanism, e.g. by permitting the light guide portion(s) 1676 to extend through the opening such that its end surface faces the other member or by permitting radiation to pass through the opening 4080. The other member (not explicitly shown) may be the dial sleeve or number sleeve which is selectively rotationally locked to member 4000. The other member expediently has angularly separated reflective encoding portions which, when rotating relative to member 4000, can excite the motion sensing unit 1200 to generate motion signals via light reflected by the reflective portions and transmitted to the motion sensing unit sensor(s) via the light guide portion(s) 1676.

As is apparent from the representation in FIG. 3F, the electronic system 1000 can be fitted in two different orientations, which are offset by 180°, to the member 4000. For example identical arrangements of openings 4080 are provided at locations diametrically opposite from one another.

The core functionality of the electronic system in conjunction with the drug delivery device unit may be as follows. The electronic system 1000, in the state when it is not connected to the drug delivery device unit has no or a very low power consumption, e.g. less than or equal to 1 μA. Specifically, the user proximity detection unit 1330, the motion sensing unit 1200, and/or, if applicable, the communication unit 1500 (not explicitly shown) may be inactive and the electronic control unit 1100 may be either in a state with very low power consumption or switched off entirely. For putting the system to use, the electronic system 1000 may be fitted to the drug delivery device unit. In the course of this connection, the connection detection unit 1700 may cause that the use detection unit 1300 and/or the user proximity detection unit 1330 is activated, e.g. by triggering switch 1710. The electronic control unit may be switched to a state of low power consumption by the connection detection unit such that the control unit can receive the signal and control the further operation of the system. Hence, after connection of the system (as an add-on module) to the drug delivery device unit, the sensor of the user proximity detection unit, such as a capacitive sensor, is preferably active and monitors proximity of the user to an exterior operation surface such as the setting surface 1610 of the user interface member 1600. As opposed to the previous state, the power consumption may have been increased, e.g. to between 3 μA and 100 μA, where 3 μA is typical for a polling frequency of 4 Hz and 100 μA is typical for a polling frequency of 100 Hz. The motion sensing unit and the communication unit may be still inactive. Thereafter, the user may set or dial a dose by rotating the user interface member 1600 up to the desired dose. As the user touches the setting surface during dose setting, proximity of the user to that surface is detected by the user proximity detection unit 1330, which triggers the use signal which causes the electronic control unit to switch the motion sensing unit 1200 on and, optionally, the communication unit. Then, the radiation sensors and radiation sources of the motion sensing unit are active and can sense relative rotational movement between member 4000 and another member of the drug delivery device unit. As opposed to the previous state, the power consumption may have been increased again, e.g. to about 900 μA when the electronic control unit, the motion sensing unit and optionally the communication unit are fully woken. After the motion sensing unit has been activated, the electronic control unit 1100 may be configured to ignore further proximity or use signals of the user proximity detection unit. When dose setting has been completed, the user presses on the delivery surface of the user interface member, i.e. its proximal surface, and moves the user interface member in the distal direction, e.g. relative to the housing 10 and/or relative to the other member to which the user interface member 1600 is rotationally locked during dose setting. In case a delivery senor is provided this sensor could be used to initialize or power up the communication unit 1500. During the initial part of the movement of the user interface member, a clutch of the dose setting and drive mechanism of the device unit may be switched—e.g. engaged or released—to switch the mechanism into a driving configuration for the dose delivery operation from a dose setting configuration. During the delivery operation the user presses on the user interface member 1600 in the distal direction, where this movement is converted into movement of the piston rod in the distal direction which may be used to drive a bung of a cartridge distally in order to expel drug or medication from the cartridge. The relative movement between the user interface member and the other member of the dose setting and drive mechanism, e.g. the dial sleeve or number sleeve, is quantified using the signals generated by the motion sensing unit e.g. by the electronic control unit or another entity which is capable of calculating dose information from the signals.

The calculated dose information, after the delivery operation has been completed, may be transmitted to another device such as a computing device, e.g. a computer, tablet, notebook, or mobile phone by the communication unit 1500, which may retrieve the information from a memory unit of the electronic system. Preferably, the communication unit is coupled or coupleable to the other device. The transmission may be wireless. In the other device, the information may be stored, e.g. to provide a dose log. After the delivery operation has been completed, the entire electronic system may be switched off, e.g. by means of a timer unit which has also been discussed already. Also, if within a certain time interval, e.g. 30 s, after the motion sensing unit has been woken, no delivery operation is detected by the motion sensing unit, e.g. no motion signal is generated, the system can be switched off again. Then, again, only the use detection unit or user proximity detection may be active.

In the system discussed above, the user proximity detection unit/the use detection unit is always active when the system is fitted to the device unit or integrated into the device. Consequently, there may be a constant power drain as the use detection unit/user proximity detection unit has to operate consistently. Below, some options are discussed on how to reduce the power consumption of the system, e.g. by only selectively activating or waking the unit when a dose setting operation or a dose delivery operation is expected to happen.

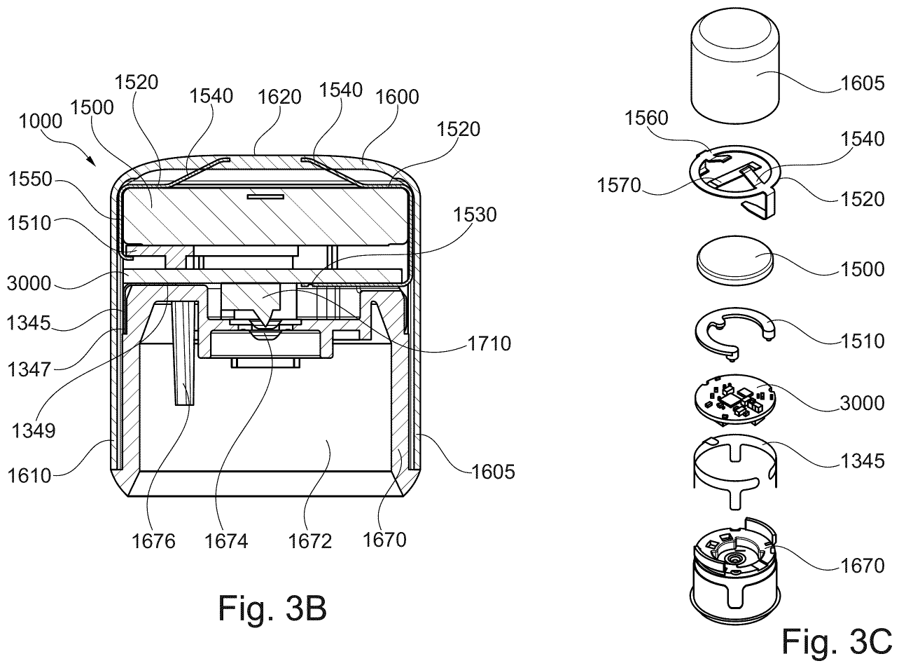

FIGS. 4A and 4B illustrate another embodiment of an electronic system 1000. The embodiment is very similar to the one discussed in conjunction with FIGS. 3A to 3F. Hence, features which are disclosed in connection with the latter embodiment do also apply for the present embodiment and vice versa. Specifically, the user interface member 1600 is shown in a somewhat transparent view in FIG. 4A. This embodiment, again, relies on a capacitive senor (not explicitly shown) for the use detection unit or user proximity detection unit. FIG. 4B shows the sensor electrode 1345 associated with the capacitive sensor. The electrode 1345 also has axially oriented and angularly separated electrode portions 1347, e.g. at least three portions or four portions as depicted. The electrode 1345 is arranged below an external surface of the user interface member, such as, in the present embodiment, the setting surface 1610 of the user interface member. The electrode 1345 is the sensing electrode of the capacitive sensor. By way of the capacitive sensor it can be detected, whether a finger of the user is close to the electrode 1345. In response thereto, the use signal or proximity signal can be generated. The capacitive sensor may be a (pure) setting sensor, a (pure) delivery sensor, or a setting and delivery sensor. That is to say it may be configured to detect user proximity to or touch of the setting surface 1610 (setting sensor), the delivery surface 1620 (delivery sensor), or both of these surfaces (setting and delivery sensor). The sensor may be configured to distinguish between which one of the delivery surface and the setting surface the user is close to or touches or not allow this distinction. A distinction between events on the setting or the delivery surface is possible, for example, if separate channels (one for the delivery surface and one for the setting surface) are provided in one sensor (e.g. in the chip or controller of that sensor) or separate sensors are provided for the delivery surface and the setting surface. The electrode portions 1347 are circumferentially distributed, e.g. equidistantly, so as to be arranged below different areas of the exterior operation surface of the user interface member 1600, e.g. the setting surface 1610. The setting surface 1610 may be formed by a grip portion of the user interface member 1600, e.g. its body 1605, which forms the exterior surface. The axial extension of the portions 1347 may be adjusted to the axial extension of the setting surface so as to enable a reliable detection of a setting operation along the entire surface. The angular separation and the angular extension of the portions 1347 is expediently chosen such that it enables a reliable monitoring of the exterior operation surface with regard to user proximity or touching by the increase in capacitance at the electrode 1345. The elongate portions 1347 extend away from a ring portion 1348 of the electrode 1345, e.g. in the distal direction. The ring portion 1348 may be closed in the circumferential direction. In one or more regions of the electrode 1345, e.g. between two adjacent electrode portions 1347, a connection portion 1349 is preferably provided, e.g. between each pair of adjacent electrode portions 1347 one connection portion 1349 may be provided. The respective connection portion 1349 may be radially and/or inwardly oriented. The respective connection portion is configured to electrically conductively connect the electrode with the sensing portion of the sensor, e.g. comprising a chip, and/or serves for fixing the electrode 1345 within the user interface member 1600. A capacitive sensor (chip) which could be used in the context of the present disclosure as capacitive sensor is Azoteq IQS 228-AS.

In the depicted configuration, the capacitive sensor is configured to generate a setting signal when the user touches the setting surface 1610 or is close to it. This signal can be used as use signal to cause activation of the motion sensing unit, e.g. by switching the radiation or light emitters and the radiation sensor from the first state into the second state.

It can be expected that the time between the initiation of the setting operation and the commencement of the delivery operation is comparatively high, especially when compared with the time between the touching of the delivery surface and the commencement of the delivery operation. This is, inter alia, due to the fact that setting the dose by rotating the user interface member may take some time and the user has to switch to another surface for initiating the delivery operation. Therefore, the response rate or frequency with which the setting sensor is operated or polled can be smaller than in the case where the delivery signal is used as use signal, especially without jeopardizing the reliability of the system. For example, frequencies or response rates of the capacitive sensor may be less than or equal to one of the following values: 10 Hz, 9 Hz, 8 Hz, 7 Hz, 6 Hz, 4 Hz, 3 Hz, 2 Hz, 1 Hz. The frequency or response rate may be between 1 and 10 Hz for example, e.g. 2 Hz or 4 Hz. The typical minimum time between the commencement of the setting operation and the associated delivery operation for delivering the set dose is in each case greater than 0.5 seconds, which is why frequencies or response rates as low as 2 Hz do still provide very good results.

In view of the capacitive sensor requiring electrical power for a sensing event, which may be performed with the frequency mentioned above, it may be advantageous to integrate another wake-up procedure into the system such as applying a vibration sensor which is used to generate or trigger a wake-up signal as has been described further above already. A suitable sensor may be the vibration sensor Kyodo CMN 200, for example.

It should be noted that it is not necessarily the case that the capacitive sensor is only a setting sensor. Rather, the same operational principle could also be implemented for a delivery sensor which has the electrode on the delivery surface. In fact, the capacitive sensor may monitor the setting surface and the delivery surface, e.g. in different channels or operation modes, where the frequency or response rate for the delivery surface is preferably higher than for the setting surface as has been detailed further above already.

The electrode 1345 of the sensor could be formed with a metal pressing, a copper tape or an (e.g. additional) flexible conductor carrier, such as a printed circuit board. The respective element for the electrode may be located on the outer diameter or on the proximal face of the electronic system, preferably below a grip or contact portion of the user interface member which is going to be contacted by the user. However, it is also conceivable to dispense with separate continuous grip or contact portions. In this case, the electrode might be directly accessible on the exterior operation surface.

Accordingly, in this embodiment, before the setting operation is performed, the user proximity detection unit/use detection unit is woken and switched from a first state to a second state of higher power consumption by the wake-up unit comprising a wake-up sensor, such as the vibration sensor (not explicitly shown). In the first or idle state the power consumption of the sensor or the entire unit may be zero. In the second or operative state, the use detection unit or user proximity detection unit may have a power consumption of less than or equal to 5 μA, e.g. 3 μA, if the setting surface is monitored to generate the use signal and more than 50 μA, e.g. 100 μA, if the delivery surface is monitored to generate the use signal. The power consumption of the capacitive sensor when operational may be between 3 μA and 100 μA, for example. The power consumption of the wake-up sensor is smaller than the power consumption when the use detection unit/user proximity detection unit is operative. In case no (setting) operation or use signal is detected within a predetermined time, e.g. within 5 seconds or less, such as within 3 seconds, after the use detection unit or user proximity detection unit has been activated by the wake-up unit, the detection unit may be switched back to the first state again.

Vibrations which are detected by the wake-up unit cause generation of a wake-up signal which, preferably directly, causes the use detection unit or user proximity detection unit to become operational. The vibration sensor may provide an interrupt to a power supply circuit for the capacitive sensor. When the sensor is triggered the power may be supplied to the use detection unit or the user proximity detection unit, respectively.

Alternatively, the electronic control unit may wake the use detection unit or user proximity detection unit in response to the wake-up signal. The vibration sensor triggers the wake-up procedure for the use (r proximity) detection unit due to vibrations detected by the sensor which trigger an electrical wake-up signal.

The vibrations, which may excite the vibration sensor, are expected to occur when the electronic system and/or the drug delivery device is handled by the user. The vibration sensor may be used as an interrupt to wake the capacitive sensor. Consequently, the capacitive sensor is woken before the dose setting or dialing operation occurs. When the user touches the setting surface, this is expected to most likely occur at the point of dose setting when the user is required to provide force or torque to the setting surface. Consequently, upon detection of the user's finger or thumb, the capacitive sensor causes the motion sensing unit to be initialized, or powered up in order to be able to detect rotation of components within the drug delivery device as has already been described above. In case no rotation is detected within a predetermined time interval after the motion sensing unit has been woken, such as within 30 seconds, the use detection unit and the motion sensing unit will be switched off again. The operational state of the communication unit may also be changed by the use (r proximity) detection unit in response to the (use) signal generated by the detection unit, if such a communication unit is provided.

Advantageously, in this embodiment, accidental waking of one or more electronic units can be avoided, unless a (large) capacitive load is present which can be sensed by the capacitive sensor, e.g. a capacitance exceeding a predetermined threshold and/or below a predetermined maximum capacitance. Thus, unintentionally pressing on the delivery surface (if the sensor monitors the setting surface) or forces transferred to the user interface member by other items, such as while the system is being carried in a bag, do not drain power unless the capacitive load is high enough. A (large) capacitive load sufficient to wake one or more electronic units may be a load occurring when direct contact is made with one or two fingers against the surface monitored by the capacitive sensor, e.g. the setting surface. A (low) capacitive load which would not cause waking of the system may be a load occurring when the surface is contacted with an, e.g. flat, metallic object, such as a house key. Moreover, vibrations while carrying the system around would not cause the motion sensing unit being switched on. During storage there are usually no vibrations such that, in this case, power drain by wake-up signals generated by the vibration sensor can be avoided entirely. However, as the power required to operate the vibration sensor is significantly lower (e.g. less than 0.5 times the, preferably minimum, power consumption of the capacitive (setting) sensor) than the one for the capacitive sensor, accidental wake-up signal generation is still tolerable. Alternatively or additionally the wake-unit may be supplied only with power when the connection detection unit detects connection to the device unit. In this case, the connection detection unit does not switch the use detection unit into an operational state but the wake-up unit. The vibration sensor preferably has a quiescent current draw (power consumption) less than 1 μA. The capacitive sensor may have a quiescent current draw of 3 μA-100 μA, dependent on its mode of operation or the polling frequency, e.g. whether proximity or touching of the setting surface and/or of the delivery surface is being monitored by the sensor. Monitoring the delivery surface usually requires a higher polling frequency (e.g. more than 80 Hz) with an associated higher power consumption.

We note again that, instead of having the setting surface monitored by the user proximity detection unit, the delivery surface may be monitored as well as an alternative or in addition to the setting surface. Here, the provision of a (low-power) wake-up unit is also advantageous since the frequency or response rate with which the sensor is operated should be higher than for the setting surface given that touching the delivery surface is very close to the start of the delivery operation and the motion sensing unit should measure the delivered dose as precisely as possible.

The capacitive sensor can also able to detect touching of the delivery surface of the electronic system as noted already. This surface may be used to induce dose data transmission or synchronisation with a program or app on another device, preferably when the surface is contacted when no dose has been set or no setting signal precedes the delivery signal generated by touching the delivery surface. In the event that the delivery surface of the user interface member is pressed (but no dose has been selected) for a time greater than a first predetermined value (e.g. 1 second) and preferably less than a second predetermined value (e.g. 5 seconds), this could be used to initiate the dose data transmission or synchronization sequence. If two channels are provided in the motion sensing unit, each channel with one optical emitter-sensor pair (IR-LED and IR sensor) which are out of phase relative to the rotating encoder, only one channel of the two channels may be observed to go to a “high” state (higher signal) or generate a characteristic signal, e.g. due to the sensor arrangement in the user interface member being moved closer to the encoder surface due the user moving the user interface member distally towards the housing and the member monitored by the motion sensing unit by pressing on the delivery surface. The characteristic signal could be used to initiate a dose data transmission or synchronisation sequence.

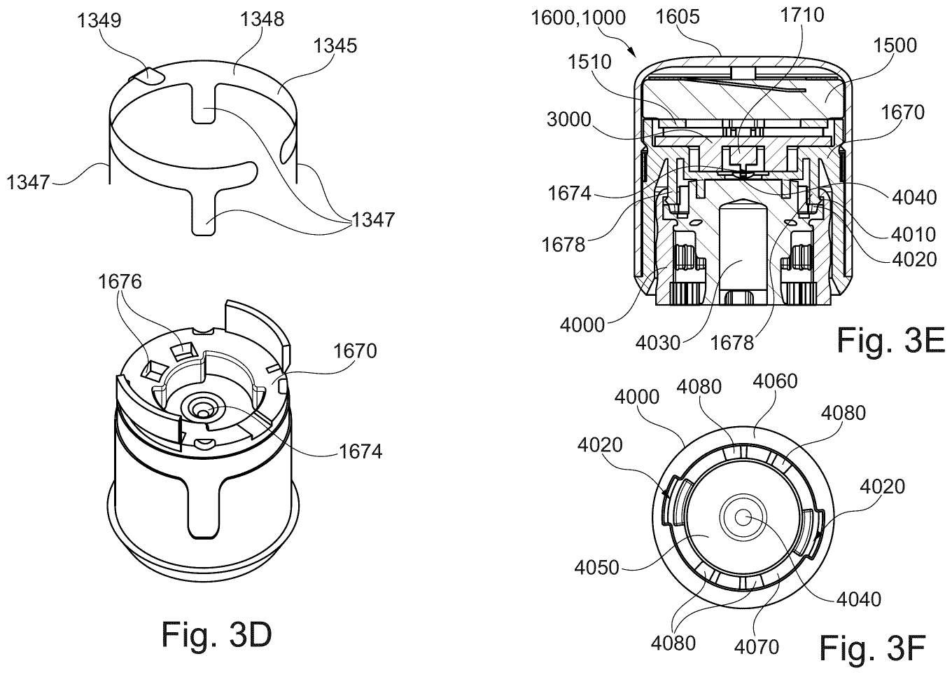

FIGS. 5A to 5C schematically illustrate another embodiment of the electronic system. As in the previous embodiment, the electronic system has a user proximity detection unit, which generates or is configured to generate a proximity signal, e.g. the use signal, when the user is close to or touches the exterior operation surface, which, in the present case, is the setting surface and/or the delivery surface of the user interface member 1600. This embodiment largely corresponds to the ones discussed previously. Accordingly, features disclosed in conjunction with these embodiments do also apply for the present embodiment and vice versa.

The user proximity detection unit comprises a sensor, which operates purely electrically, i.e. it does not require mechanical moving parts in order to produce or trigger the signal, such as to indicate a sensing event. In the present case, the user interface member 1600 comprises a contact or contactless sensor or switch in the interior the user interface member 1600. The sensor is a contactless sensor, e.g. a capacitive sensor as in the previously discussed embodiments. In the depicted embodiment, the sensor 1650 e.g. a sensing element thereof such as the sensor electrode discussed above, is associated with the delivery surface, e.g. arranged below the delivery surface 1620, in the interior of the user interface member 1600. Using a contactless sensor facilitates having a tightly sealed user interface member without any protruding portions of either a sensor or sensing surface or movable member on the exterior of the user interface member which needs to be sealed additionally. This arrangement is shown in FIG. 5B. FIG. 5A, shows the general arrangement of the user interface member 1600 relative to the housing 10, either as an add-on to the device unit comprising the housing 10 or integrated into device.

In addition to the delivery surface 1620, the setting surface 1610 may also be provided with a user proximity detection unit. For this unit, also a non-contact or contactless sensor may be used in a similar setting as the delivery sensor in FIG. 5B as setting sensor 1660, see FIG. 5C. The sensor may be a capacitive sensor as well. However, also other arrangements or configurations may be possible.

Accordingly, when the delivery surface is touched, a signal may be generated, e.g. a delivery signal by the delivery sensor. When the setting surface is touched, a setting signal may be generated, e.g. by the setting sensor.

Capacitive sensors constantly drain power while they are in operation as noted above. The setting sensor 1660 can be used as a wake-up unit to wake up the delivery sensor 1650 associated with the delivery surface 1620. The delivery sensor can be part of the use detection unit or the user proximity detection unit. If one or both user proximity detection units are contained in the interior of the user interface member, provision of a tightly sealed user interface member is facilitated. Also, it is facilitated to provide the user interface member as an add-on to an existing drug delivery device unit such as a clip-on to a dose setting and/or injection button of the device unit, for example.

In case two contactless, e.g. capacitive, sensors are used, one for the setting surface and one for the delivery surface, it may be advantageous to operate the setting sensor with a frequency or response rate which is lower than a frequency or response rate with which the delivery sensor is operated, especially, if the setting sensor is used as a wake-up sensor for the delivery sensor. For example, the frequency or response rate of the setting sensor may be at least 5 times, 10 times, 15 times, 20 times, 30 times, 40 times, or 50 times lower than the one of the delivery sensor. In other words, f_{S}/f_{D} may be less than or equal to one of the following values: 1/5, 1/10, 1/15, 1/20, 1/25, 1/30, 1/35, 1/40, 1/50. Here f_{S} denotes the frequency or response rate with which the setting sensor or switch is operated and f_{D} denotes the frequency or response rate with which the delivery sensor or switch is operated. Operating the respective sensors or switches requires at least temporarily supplying power from the power supply to the respective sensor for performing one sensing operation which checks, whether the user is proximate to or touches the respective surface. Thus, user proximity to the setting surface may be detected with a small frequency or response rate, i.e. the sensor may be polled less often, than the user proximity to the delivery surface. If a setting event is sensed, the use signal may be triggered to render the delivery sensor operational e.g. by the electronic control unit. For example, the frequency f_{S} with which the setting sensor or switch is polled or operated may be less than or equal to one of the following values: 10 Hz, 8 Hz, 5 Hz, 4 Hz, 3 Hz, 2 Hz. Alternatively or additionally, the frequency f_{D} with which the delivery sensor or switch is polled or operated may be greater than or equal to one of the following values: 10 Hz, 15 Hz, 20 Hz, 25 Hz, 30 Hz, 40 Hz, 45 Hz, 50 Hz, 60 Hz, 70 Hz, 80 Hz, 90 Hz, 100 Hz. Accordingly, the setting sensor may be operated or polled five times per second or less, e.g. twice per second or four times per second, in order to check, whether the user is in proximity to the setting surface. The associated power consumption is comparatively low, preferably less than or equal to 15 μA as discussed further above already, e.g. 3 μA. As opposed to this, the delivery sensor or switch, may be operated or polled at a higher frequency, e.g. 10 times per second or more such as up to 100 times per second. The power consumption is accordingly higher, e.g. about 100 μA, as has been discussed above already.

In this embodiment, the setting operation or an event indicative for the setting operation like the user touching the setting surface can be used to wake up the use detection unit, e.g. the delivery detection unit, in particular the associated, e.g. contactless, sensor or switch. Also, any remaining components of the electronic system which may be needed for the operation of the delivery detection unit may be switched to a state of higher power consumption or woken up. However, it is not yet required to wake up the motion sensing unit which may have a particularly high power consumption on account of the optoelectronic components (emitter and sensor/detector) used in this unit, for example, as well as potentially the communication unit. Expediently, the motion sensing unit is switched to the state of higher power consumption, e.g. by the control unit, only if the delivery detection unit detects that the user is close to the delivery surface. Detecting a user touching the delivery surface, preferably only together with the preceding setting event, hints to a delivery operation, which is to be expected and, consequently, it is worth to supply the motion sensing unit with power. Similar considerations apply for the communication unit or other units which are not required for the setting operation. Furthermore, it is possible that only a delivery detection unit or only a setting detection unit is provided.

In this embodiment, the electronic system may be switched on in two stages. Firstly, the electronic module is woken from its dormant or idle state during dose setting (dialling), but the motion sensing unit is not initialised at this stage, e.g. via the setting sensor 1660.

A capacitive sensor (the delivery sensor 1650) is woken by the dialling event, and is able to detect the presence of the user's finger/thumb at or in close proximity to the delivery surface 1620 of the user interface member. It is expected this event will most likely to occur at the point of dose delivery, when the user is required to provide an axial force on this top face.

When the user contacts the delivery surface of the electronic system to initiate dose delivery, the capacitive sensor detects the presence of the finger/thumb and may cause initialisation of the motion sensing unit, e.g. the IR-LED detection system, to detect rotation of components within the device during dose delivery. If no rotation is observed by the motion sensing unit, e.g. within a predetermined time, such as 30 seconds, the capacitive sensor will be switched off.

This concept utilises the fact that the power consumption of a capacitive sensor is significantly lower than the power consumption of the motion sensing unit, thereby lowering the overall power consumption of the electronic module by limiting the on-time of the motion sensing unit to during dose delivery only. Typical quiescent current draw for capacitive sensors are: at 4 Hz polling frequency about 3 μA, at 100 Hz polling frequency about 100 μA. The motion sensing unit can have a power consumption of more than 500 μA when it is operational.

Additionally, this concept limits accidental waking of the electronic system. If the delivery face/proximal face of the electronic system/user interface member is unintentionally pressed (for example, compressed by other items while carried in a bag) it would not cause the motion sensing unit to wake. Rather, only dose setting followed by finger/thumb contact on the delivery surface of the system would cause the motion sensing unit to be initialised.

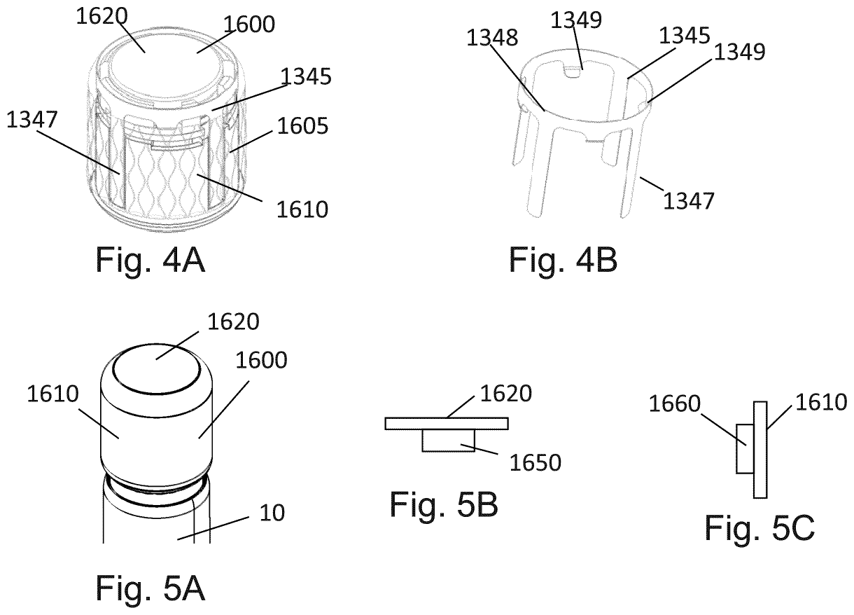

FIGS. 6A, and 6B illustrate an embodiment of an electronic system, especially a user proximity detection unit which can be used in the use detection unit of the system or in other functionalities as the user proximity detection unit. With respect to the general functionality, this embodiment is similar to the embodiments which have been described above and it also applies an electrical user proximity detection unit or use detection unit. Accordingly, features disclosed in conjunction with this embodiment do also apply for the other embodiments and vice versa. However, as opposed to the other embodiments, the present embodiment relies on a contact sensor or switch, where the user has to make contact with a sensitive surface to trigger the sensor or switch.

FIG. 6A shows a side view and FIG. 6B shows a top view on a schematically sketched portion of a user interface member 1600. The user interface member 1600 is movably connected to a housing 10, e.g. of the drug delivery device (unit). The depicted user interface member is a combined dose setting and dose delivery member such that the member is operated by the user during the dose setting operation and during the dose delivery operation. Consequently, it has two different surfaces, the setting surface 1610 and the delivery surface 1620 as discussed above. The setting surface 1610 may be a side surface which delimits the user interface member 1600 laterally and/or faces in the radial direction. The radial direction may be relative to axis A, which may be a main longitudinal axis of the device, the housing, or the user interface member and/or run between the distal and proximal ends of the respective component. Axis A may be the rotation axis of the rotation of the user interface member relative to the housing during dose setting. FIG. 6B shows the top view onto the proximal setting surface 1620. As opposed to one component which has two different surfaces, the user interface member for dose setting and the user interface member for dose delivery may also be different as has been discussed above already. The user interface member comprises a user interface member body 1605, which provides the setting surface and/or the delivery surface. The body may retain one or more electrical or electronic components of the system as has been described previously in its interior.

One or more first electrical contacts or contact surfaces 1630 are arranged on the setting surface 1610 and/or the delivery surface 1620. Furthermore, one or more second contacts or contact surfaces 1640 are provided on the setting surface and/or the delivery surface. The contacts 1630 and 1640 or contact surfaces are electrical contact surfaces and/or electrically conductively connected to the electronic control unit, which is provided in the interior of the user interface member, either directly or indirectly. The first contact surfaces and the second contact surfaces may be surfaces of different electrical potentials, e.g. of opposite polarities. The first contact surfaces 1630 are preferably equipotential surfaces as are the contact surfaces 1640.

When the user operates the user interface member, the first contact surfaces 1630 and the second contact surfaces 1640 are arranged such that the user will touch a section of the first contact surfaces and a section of the second contact surfaces simultaneously during dose setting and/or during dose delivery, on the setting surface 1610 and/or on the delivery surface 1620 respectively. In other words, the pattern of the first contact surfaces and the second contact surfaces on the setting surface may be configured such that the user will always touch a section of both contact surfaces on the setting surface when performing the dose setting operation. Alternatively or additionally, the pattern of the first contact surfaces and the second contact surfaces on the delivery surface may be configured such that the user will always touch a section of both contact surfaces on the delivery surface when performing the dose delivery operation. Thus, when commencing the respective operation, the user will provide a bridge between the first contact surfaces and the second contact surfaces such that the contact surfaces are conductively connected through the user's finger.

The resistance which is contributed by the user is comparatively high, such as from 1 kΩ to 200 kΩ. When the user bridges the contacts, the resulting change in the resistance may be used to cause the switching of one of the units of the electronic system from the first state into the second state where the power consumption is higher. The flowing current may be the use signal/proximity signal. The event that the user touches the contact surfaces can be used to trigger a dedicated circuit which may comprise a transistor such as a MOSFET, for example, in order to activate further electronics in the system, e.g. the electronic control unit 1100, which may then switch on the motion sensing unit 1200 and/or the communication unit 1500. The dedicated circuit may hold the activation of the electronic control unit, even if the user does no longer touch the contact surfaces. Consequently, the circuit may be a hold circuit. The dedicated circuit in addition to the electronic control unit may have an optimized power consumption as compared to using the control unit itself to evaluate whether the contact surfaces are bridged.

After the desired operation has been performed an end-of-operation signal may be generated, e.g. by a component reaching a home position triggering an according switch. The electronic control unit, in response to the end-of-operation signal can issue a signal, such as an off signal, to the dedicated circuit which then may be deactivated in response to the off signal. When a user again touches a section of two contact surfaces 1630 and 1640 simultaneously, the hold circuit may resume its operation and, again, activate the respective unit such as the electronic control unit, the motion sensing unit and/or the communication unit, for example. Such a dedicated circuit and its arrangement are shown schematically in FIG. 6C.

Instead of providing a dedicated circuit to hold the electronic control unit active, the current generated by bridging the contacts can be used to directly trigger the switching of the electronic system to the state of higher power consumption by the electronic control unit. Switching off the electronic unit(s), without the dedicated circuit, may be achieved by another unit, e.g. the timer unit and/or the electronic control unit. The dedicated hold circuit may, however, provide the more power efficient solution.

The first contact surfaces on the setting and the delivery surface are conductively interconnected. The same holds for the second contact surfaces on the setting and the delivery surface. Alternatively, the first contact surfaces and/or the second contacts on the delivery surface 1620 are electrically separate from the associated contact surfaces on the setting surface 1610. Specifically, it can be distinguished whether the user touches only the setting surface, only the delivery surface or both surfaces. In this way, the setting and delivery operation can be distinguished from one another more easily. It should be noted that the exemplary embodiment using a variety of contact surfaces which are strip-like, on the setting surface 1610, which, contrary to the depicted embodiment, may also be arranged in alternating fashion, and almost full circular contact surfaces on the proximal delivery surface 1620 are merely exemplary arrangements. Other arrangements are possible, such as alternating radially oriented strips of contact surfaces on the delivery surface, for example. Also, the number of strips can be adjusted.

Also, if user contact with the delivery surface and the setting surface should be monitored separately, a set of three electrically or galvanically separated contact surfaces may be sufficient already. One of these contact surfaces may extend from the delivery surface to the setting surface, i.e. this contact surface is accessible on the setting surface and on the delivery surface. Thus, this contact surface forms a common contact surface on the setting surface and on the delivery surface. The other two contact surfaces, one only accessible on the delivery surface and one only accessible on the setting surface, are electrically separated. The common contact surface may be used to detect contact with the delivery surface—in combination with the other contact surface on the delivery surface—and to detect contact with the setting surface in combination with the other contact surface on the setting surface. Thus, to distinguish the user touching the setting surface and the user touching the delivery surface, e.g. for distinguishing a dose setting operation from a dose delivery operation, three electrically separated contact surfaces may be sufficient, instead of having four separated contact surfaces, which is also an option.

The respective contact surfaces may be integrated into the user interface member and recessed relative to an enveloping surface (enveloping surface not explicitly shown). The contact surfaces may be arranged in recesses on the exterior surface. In this manner, the risk of an undesired activation of the electronics may be reduced. The users hand and the fingers have small flexibility such that the skin can even mechanically contact the contacts even in case they are recessed relative to the enveloping surface of the user interface member. The risk of accidentally bridging the contacts with rigid conductive members is reduced considerably by recessing the contact surfaces relative to the enveloping surface.

The electronic system may comprise a signal evaluation unit (not explicitly shown), which is configured to evaluate, whether the signal, e.g. the use signal, meets a predetermined criterion or a set of criteria. If the signal does not meet the criterion or the set of criteria, the signal evaluation unit may feed this result back to the electronic control unit, which, in this case does not switch one or more of the other units to the state with higher power consumption. Such a unit may be implemented not only in this embodiment but also in other embodiments. In the present embodiment, the signal evaluation unit may use the magnitude of the current flowing through the user as a criterion, which, knowing the voltage of the power supply, gives a measure of the electrical resistance or resistivity of the element bridging the contacts. If the resistance or the resistivity is less than the resistance or resistivity characteristic for human tissue/skin bridging the contact surfaces 1630 and 1640, the signal, which is generated, is regarded as not being generated by a user and, consequently, the power consumption is not raised as a setting and/or delivery operation is not to be expected.

The embodiment which has been discussed above has a variety of advantages. For example, it is comparatively easily realized and implemented in electronics. It also does not involve moving parts and does not rely on movement of parts of the drug delivery device or the system relative to one another. The embodiment works for both operations, dose setting and dose delivery.

Moreover, the use signal is generated before the user interface member is moved for dose setting and/or dose delivery. Therefore, the units of the electronic system which need to be woken or powered on, have sufficient time to be ready for operation when the mechanical operation of dose setting and/or dose delivery is being commenced. The electrical contact surfaces, e.g. of metal or another electrical conductor, may be integrated into the user interface member body of plastic during a moulding process, e.g. injection moulding.

The contact surfaces 1630, 1640 are part of a resistive touch sensor or switch, which requires mechanical contact with the contact surfaces to be triggered.

Of course, other options for touch sensors or switches exist which can detect the proximity of the user to a surface of the user interface member based on mechanical contact with a sensing surface of the sensor or even contactless. One example for an alternative resistive sensor or switch is a pressure-sensitive resistor network. It is preferred that the setting surface and/or the delivery surface are provided with, preferably separate, touch sensitive surface areas.

The contact surfaces 1630 and 1640 are accessible on the exterior surface of the user interface member 1600 and can be used to charge a rechargeable battery as power supply. A dedicated charging bay configured to receive the user interface member may be provided for charging.

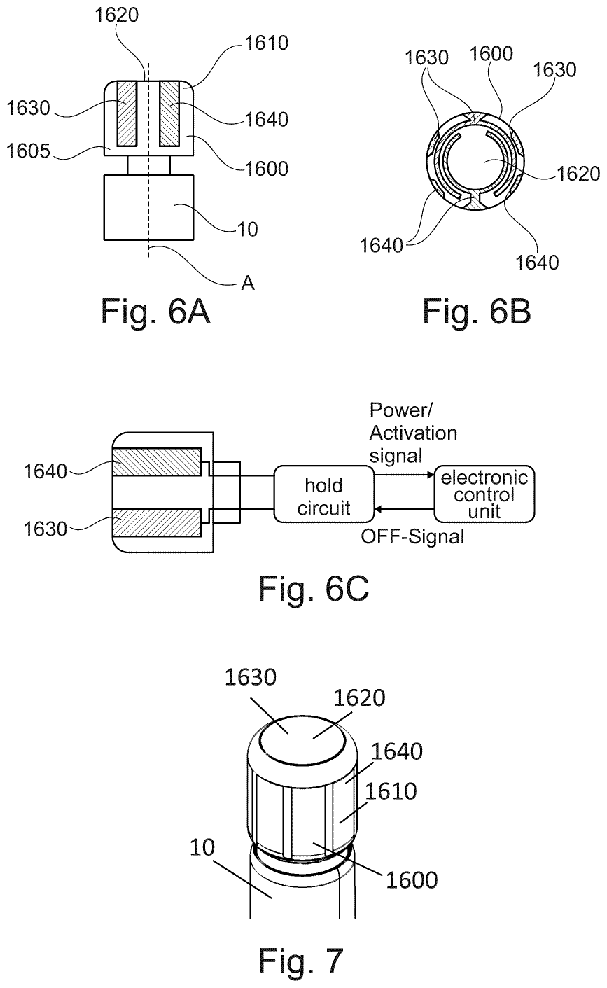

FIG. 7 illustrates an embodiment similar to the previous one, where the surface(s) of the user interface member 1600 is/are provided with or have touch sensitive surface areas, e.g. formed by electrical contacts, such as contact pads, or other touch sensitive sensors. The touch sensitive surface areas on the setting surface may be evenly distributed circumferentially, e.g. by contact pads of alternating polarity or electrical potential (a higher potential surface is followed by a surface of lower potential and vice versa) or the entire surface may be touch sensitive. The touch sensitive surface area of the delivery surface 1620 may be formed by one continuous touch sensitive surface area or by separate surface areas, e.g. by contact surfaces of different electrical potentials.

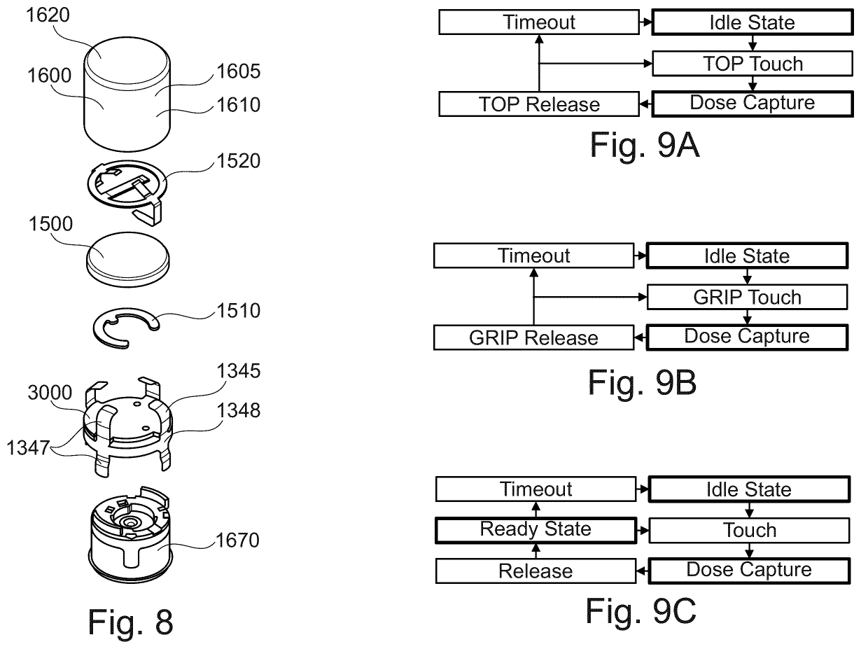

FIG. 8 shows an exploded view of a further embodiment of the electronic system. This embodiment is similar to the one described in conjunction with FIG. 3. Mainly differences to the embodiment depicted in FIG. 3C are pointed out in the following. The electronic system 1000 comprises a conductor carrier 3000 (e.g. a PCB, printed circuit board). The conductor carrier 3000 comprises a user proximity detection unit with a sensing element (not explicitly shown, e.g. a microcontroller or chip). The sensor electrode 1345 of the user proximity detection unit is conductively connected with the sensing element, preferably via conductors of the conductor carrier. The electrode 1345 has a plurality of electrode portions 1347, e.g. four portions in the depicted embodiment. The electrode portions may be axially oriented, e.g. largely axially oriented. The electrode portions may be oriented parallel to one another. The electrode portions 1347 are connected with each other via a ring-like portion 1348. The ring may be open with two ends which face one another in the angular direction or closed in the angular direction. The ring-like portion 1348 may surround the conductor carrier 3000, e.g. extend along a lateral edge of the conductor carrier 3000. The electrode 1345 can be affixed to the conductor carrier 3000 and/or conductively connected to the conductor carrier, e.g. via the ring-like portion 1348. The electrode portions 1347 extend from the ring-like portion 1348 in a distal direction. The electrode portions 1347 extend from the ring-like portion 1348 in a proximal direction. In other words, the ring-like portions can extend in opposite axial directions as seen from the ring-like portion. In a proximal end region, the electrode portions 1347 extending in the proximal direction may curve inwardly or be bent inwardly. In this way, the electrode portion may face towards the surface delimiting the user interface member body 1605 proximally in its proximal end region. The electrode may thus act as a sensing electrode for the delivery surface. In the proximal end region, the electrode portions may point towards a center axis (main longitudinal axis). In the proximal end region, the proximally extending electrode portions 1347 may comprise surfaces parallel or substantially parallel to the delivery surface 1620 of the user interface member when assembled. The shape of the proximally extending portions 1347, especially in the proximal end region, may conform to an inner surface of the user interface member body 1605 (e.g. cap, button). At least the end regions of the proximally extending electrode portions 1347 may be used to monitor proximity to the delivery surface. The ring-like portion 1348 and/or the distally extending electrode portions 1347 may be used to monitor proximity to the setting surface. The user proximity detection unit with the depicted electrode may not distinguish which particular surface is being touched by the user. Alternatively, the electrode may be connected to different sensing elements for the setting surface and the delivery surface to distinguish between setting and delivery proximity or touch events being detected via a single electrode. Separate electrodes are possible as well, of course.

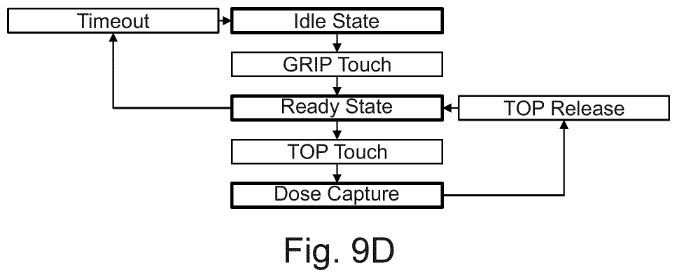

FIGS. 9A to 9D by way of the schematic process flows in FIGS. 9A to 9D illustrate embodiments of a method of operation of and/or for any one of the previously described electronic system 1000, e.g. the one in FIG. 2 to 5 or 8 which may employ capacitive sensors for monitoring proximity to the user interface member. The electronic systems or drug delivery devices described further above and below may be configured to operate according to the methods. Hence, features disclosed for the methods do also apply for the system (and the drug delivery device) and vice versa.

In a first step, the electronic system (e.g. an add-on module) may be attached to a drug delivery device unit, e.g. for an injection device (such as a pen-type injector). During or after attachment, the user proximity detection unit may become operational or be activated, e.g. via the connection detection unit 1700 which has been described further above. Alternatively, the system may be integrated into the device and/or the user proximity detection unit may be activated separately or be continuously active. The user proximity detection unit is configured to detect proximity or touch with respect to at least one of the setting surface and the delivery surface or both.

The electronic system 1000 is in a first state (or idle state) when the (respective) user proximity detection unit is active, e.g. after attachment to the device unit or after activation of the user proximity detection unit. In this state, one or more user proximity detection units are operational (e.g. powered) to detect proximity of the user with respect to or the user touching at least one of or both of the delivery surface 1620 (e.g. touch at the top or proximal surface) and the setting surface 1610 (e.g. touch at the grip or lateral surface) of the user interface member 1600 (or its body 1605). However, the motion sensing unit 1200 may not be active or operational, e.g. to generate motion signals, in the first state. The same may hold for the communication unit 1300 which may not be active or operational in the first state.

As will be apparent from the disclosure further above, there are several configurations for the user proximity detection. The system may be configured to detect proximity or touch events just for the delivery surface (top surface) or just the setting surface (grip or lateral surface). This can be done with one user proximity detection unit monitoring the respective surface. Alternatively, the system may be configured to detect proximity or touch events for both surfaces. In this case there are several options. One option is that the system is configured such that it cannot distinguish between the surfaces at which the event is detected. Another one is that the system can distinguish which surface has the user in its proximity or is being touched by the user (i.e. the system can decide whether the user is close to or touches the setting and/or the delivery surface). A system that cannot (or does not) distinguish between the events for the two surfaces can be provided by one sensing electrode having at least one portion assigned to the setting surface and at least one portion assigned to the delivery surface, e.g. along with one electronic sensing element, e.g. a sensor chip, to which the sensing electrode is operatively connected (see the electronic system and in particular the electrode depicted in FIG. 8, for example). If a distinction should be made between the surfaces for the touch or proximity event a multi-channel sensor chip, e.g. in combination with different sensing electrodes, can be used or multiple sensor chips, preferably with associated electrodes, can be used with one channel or sensor chip being provided for the setting surface and another channel or sensor chip for the delivery surface.

FIG. 9A illustrates an embodiment of a method, in which touch or proximity events for just the delivery surface 1620 are detected or detectable by the electronic system. That is to say the user proximity detection unit 1330 is capable of detecting proximity to the delivery surface (and not the setting surface). Thus, the system may be a system comprising a delivery sensor (and not a setting sensor, see also the embodiments discussed further above). At the beginning, the system is in the first or “idle” state, in which touch or proximity events can be detected.

When the thumb or finger of the user is detected by the user proximity detection unit (“Top Touch” in FIG. 9A), the system is woken and the motion sensing unit (or dose capturing system) is initialized (“Dose capture”). Then, the user can deliver the dose and the delivered amount can be determined via the motion sensing unit. The communication unit may still be inactive when the motion sensing unit is operational.

A timer functionality (preferably incorporated in the electronic system) may be used to consider a dose delivery operation complete when a predetermined time, e.g. 2 s, since the detection of the touch event has elapsed. Alternatively or additionally, the signal(s) generated by the motion sensing unit may be monitored and, after a predetermined time, e.g. 2 s, has elapsed since the last signal characteristic for the dose delivery operation has been detected the dose delivery operation is considered to be complete. Alternatively or also additionally, the release of the user interface member—which may also be detectable via the user proximity detection unit (e.g. due to the change in signal from a signal characteristic for touching to one characteristic for not touching)—can be used as indicator that the dose delivery operation is complete. In this case, the dose delivery operation may be regarded as being completed when a predetermined time, e.g. 2 s, after the release of the delivery surface of the user interface member has been detected has elapsed or in direct response to the detection of the release. In case the release of the delivery surface is not used to indicate the completion of the dose delivery operation, the user may keep the finger or thumb on the user interface member during the synchronization process via the communication unit, which may increase the users confidence in the system. In case the release of the delivery surface is used to indicate the completion of the dose delivery operation, the likelihood of wrongly interpreting an interrupted dose (where the user still maintains contact with the user interface member but does not exert the force necessary for the delivery operation) as a complete dose is increased.

After the dose delivery operation has been completed or is considered complete, the motion sensing unit may be switched off. The data generated by means of the motion sensing unit may be stored in an internal memory of the system and/or an automatic data synchronization with the external device, e.g. a phone (or similar), may be initiated via the communication unit 1500.

The system may return to the first or idle state after a predetermined time has elapsed (“Timeout”), e.g. after release of the delivery surface of the user interface member (“Top Release”), after the last signal has been generated by the motion sensing unit or without any motion signal being generated by the motion sensing unit. After the predetermined time, which may be 45 s or lower, e.g. 30 s, the motion sensing unit may be switched off. FIG. 9A shows a high level process flow of this method.

A new touch or proximity event detected by the delivery sensor expediently will cause the system to initialize or activate the motion sensing unit once more (if such initialization is needed). The touch or proximity event, preferably, interrupts any pairing or synchronization activity which might be still ongoing and guarantees that any following delivered units are captured by the system. This is particularly advantageous in the case of priming of a device (which might be necessary after a new cartridge has been connected to the dose setting and drive mechanism of the device to ensure that the drug delivery device is working properly and delivers doses in the amount set during dose setting). In the case of priming, one dose delivery operation may follow quickly after another.

In order to wake the device or system, such as for the purpose of pairing with an external device or for manual synchronization, e.g. with a paired external device, the user may press the user interface member via the delivery surface (when a zero dose has been set, i.e. the user interface member is in its initial position before dose setting is commenced and/or in its end position after completion of a dose delivery operation). This will trigger the initialization of the motion sensing unit as described above, but no units will be counted. The duration of this zero dose delivery (“0U dose”) can be used to infer what action the user wishes to perform, e.g. pairing or manual synchronization, where the duration required for pairing may be greater than the one for manual synchronization as pairing is likely to occur less often than manual synchronization. This approach to pairing and manual synchronization is facilitated because the optical motion sensing unit can detect the axial position of the user interface member (dose button) relative to the drive mechanism, e.g. the number or dial sleeve (see also the descriptions further above, e.g. in connection with FIG. 3).

As the delivery surface is being monitored, the delivery sensor may have to be polled comparatively often or with a high response rate or frequency (e.g. 80 Hz, see the description further above) in this embodiment as the onset of the delivery operation should not be missed. Activating the motion sensing unit too late results in an inaccurately determined dose.

FIG. 9B illustrates an embodiment of the method, in which touch or proximity events for just the setting surface 1610 are detected or detectable by the electronic system. That is to say the user proximity detection unit 1330 is capable of detecting proximity to the setting surface (and not the delivery surface). Thus, the system may be a system comprising a setting sensor (and not a delivery sensor, see also the embodiments discussed further above). At the beginning, the system is in the first or “idle” state, in which touch or proximity events can be detected.

When the thumb or finger of the user is detected by the user proximity detection unit (“Grip touch” in FIG. 9B), the system is woken and the motion sensing unit (or dose capturing system) is initialized. The setting operation will precede the delivery operation and hence, serves as an indicator that the motion sensing unit will soon be required. The user can subsequently deliver the dose and the delivered amount can be determined via the signals generated by the motion sensing unit (“Dose capture”). The communication unit may still be inactive when the motion sensing unit is operational.

As the setting surface is being monitored the sensor may have to be polled less often or with a low response rate or frequency (e.g. 5 or 8 Hz, see the description further above) in this embodiment. The response rate may especially be lower than in the one of FIG. 9A where the delivery surface is monitored by the sensor, because there usually are some seconds between the commencement of the setting operation and the commencement of the subsequent delivery operation and the motion sensing unit has to be operational during the delivery operation only.

The motion sensing unit remains active or operational until a dose has been recorded (e.g. after generation of the last motion signal) and/or a timeout expires, e.g. less than 45 s, such as 30 s (“Timeout”). Thereafter, the device returns to its idle state until the next touch event is detected. Optionally, a sensory indication (e.g. an LED) of the system being in a second or “ready” state (e.g. a state where the motion sensing unit is operational) may be given to the user.

Synchronization after the dose delivery operation may be effected in the same manner as discussed in connection with FIG. 9A. Also, a synchronization or pairing process may be interrupted in case a new touch or proximity event is detected while the process is ongoing. FIG. 9B shows a high level flow of this method.

In order to wake the system for pairing or manual synchronization, the same principle can be used as described in conjunction with FIG. 9A. This will require the user to have made contact with the setting surface (grip surface) before pressing the user interface member (button), as this action alone will not wake the system (as there is no proximity sensing functionality for the delivery surface). Alternatively, an additional switch may be included for starting the communication unit, which may be or comprise a BLE unit.

FIG. 9C illustrates another embodiment of the method. In this embodiment, touch or proximity events for the setting and delivery surfaces can be detected. However, a distinction of which particular one of these surfaces has the user in its proximity cannot be made. The electronic system includes a single sensor that is able to detect any touch on the outside of the user interface member. This sensor is a combined setting and delivery sensor (see, e.g., FIG. 8).

At the beginning, the system is in the first or “idle” state, in which touch or proximity events can be detected. When touch of or proximity relative to the user interface member is detected, the motion sensing unit is initialized and the motion signals may be generated (“Dose capture”) during the dose delivery operation. The system is then in an intermediate or “ready” state. When the user interface member is released (which is expediently detectable via the change in the signal of the user proximity detection unit) the system remains in the intermediate or “ready” state (with the motion sensing unit being operational). The system remains in this state until a subsequent touch or proximity event occurs and/or a timeout expires, e.g. a timeout as has been described before, such as a timeout less than or equal to 45 s, e.g. a 30 s timeout. FIG. 9C shows a high level process flow of this method.

As noted above, the intermediate or “ready” state may involve leaving the motion sensing unit active or operational while the system is in this state. Alternatively, the system may be configured such that the motion sensing unit is inactive, inactivated or switched off (e.g. in response to the release of the user interface member) in this state and/or such that the sensor is set to a higher response rate in this state than in the idle state (e.g. more suitable for monitoring the delivery surface, see the description further above). As discussed in connection with FIG. 9B, a slow response rate for detecting a setting event via the setting surface is acceptable, but a faster response may be advantageous for detecting a delivery event. Since normal use requires setting to precede delivery, the sensor in the first or “idle” state may run at a low response rate.

In order to wake the electronic system for pairing or manual synchronization, the same principle can be used as described in conjunction with FIG. 9A.

FIG. 9D illustrates another embodiment of the method. In general, this embodiment is similar to the one discussed in conjunction with FIG. 9C. Hence, the following discussion focusses on the differences. In this embodiment, the system can distinguish between delivery surface (top) and setting surface (grip) touch or proximity events. This may be achieved by employing two sensing elements (e.g. two sensor chips or sensor controllers with separate electrodes) or a single sensing element (e.g. one sensor chip or controller) with capacity for two input channels (e.g. for two different electrodes). Battery life modelling suggests the benefit of reducing the time spent with the motion sensing unit being active is worth the additional quiescent current draw of an additional sensing element or channel. Thus, the user proximity detection unit may monitor the setting surface and the delivery surface. Being able to distinguish or to determine which surface of the user interface member 1600 is being manipulated by the user, i.e. the setting or delivery surface, allows for more precise handling of touch or proximity events and/or more precise control over power consumption.

A touch or proximity event on the setting surface (grip) does not immediately initialize the motion sensing unit as in the previously described embodiment. Instead this event can be used to control the response time or rate of the delivery sensor (or the associated channel), preferably to switch that sensor on or to a higher response rate. References above and in the following to a “setting sensor” should be considered as covering a separate setting sensor (chip) or one setting sensor channel of a sensor (chip). The same holds for references to a “delivery sensor” and delivery sensor channels.

In the first or “idle” state, the delivery sensor may be completely off (inactive) or active, e.g. in a slow response rate setting (e.g. equal or below 10 Hz, see also the description further above, e.g. in conjunction with FIGS. 3A to 5C). The setting sensor may be active, expediently in a slow response rate setting. The response rates of the delivery sensor and the setting sensor may be equal in the first state (if the delivery sensor is operational at all and not switched off) or different.

In an intermediate or “ready” state of the system into which the system is switched in response to a touch or proximity event detected by the setting sensor, the delivery sensor is set to a fast response setting. That is to say the response rate of the delivery sensor is increased over its response rate in the idle state and/or the response rate of the delivery sensor is set to be greater than the response rate of the setting sensor in the idle state.

In this state, events on the delivery surface may be detected with higher response rate (e.g. providing a response time of less than 10 ms, e.g. 9 ms). The setting sensor can remain operational in the ready state or be switched off. The system may be provided with a timer or delivery sensor timeout which switches the system back to the first or idle state in case the delivery sensor does not detect a proximity or touch event within a predetermined time. The predetermined time may be greater than or equal to 45 seconds, e.g. greater than or equal to 1 minute, e.g. 1 to 2 minutes. Preferably, the predetermined time for the delivery sensor timeout is greater than the one for a motion sensing unit timeout (which may be less than 45 s, such as 30 s or even less, e.g 20 s or less, or 10 s or less, such as between 3 to 5 s). The motion sensing unit timeout after which the motion sensing unit is switched off or inactivated can be less than in the embodiments previously described (e.g. in connection with FIGS. 9A to 9C), as the motion sensing unit can be initialized separately by way of a touch or proximity event detected by the delivery sensor.

The delivery sensor can be used to initialize the motion sensing unit as in the embodiment of FIG. 9A, e.g. starting from the ready state of the system. A touch or proximity event detected by the delivery sensor, when the system is in the state, initializes the motion sensing unit and the motion signals can be generated by the motion sensing unit, e.g. to generate dose data, during the delivery operation (“Dose capture”). The release of the delivery surface by the user may be detectable via the delivery sensor. After the delivery surface of the user interface member is released (“Top release”), which likely occurs after the dose delivery operation has been completed, the motion sensing unit may be kept active until the motion sensing unit timeout expires, e.g. 3 to 5 s to account for a briefly interrupted dose. The motion sensing unit can be switched off or deactivated after the motion sensing unit timeout expires (“Timeout”). Alternatively, the motion sensing unit can be switched off or deactivated in (direct) response to the release of the delivery surface. After the deactivation or switching off of the motion sensing unit, the system may be set to the ready state once again, i.e. with the delivery sensor operating with a high response rate, e.g. higher than in the idle state. Keeping the delivery sensor at the high response rate ensures that interrupted dose delivery operations (the duration of the interruption potentially exceeding the motion sensing unit timeout) are still captured quickly as the motion sensing unit is initialized with high response rates. If no motion signals have been generated by the time the relevant timeout expires, e.g. the delivery sensor timeout, or if no touch or proximity event on the delivery surface has been detected the system is switched back to the first or idle state.

As described in conjunction with FIG. 9B, the ready state will automatically expire after some time. This may optionally involve sensory feedback (e.g. via LEDs) to the user indicating that the system is no longer ready to capture motion of the dose setting and drive mechanism in the intended manner, i.e. with a high response rate entailing prompt initialization of the motion sensing unit. This embodiment is power efficient by minimizing the amount of excess time spent with the motion sensing unit activated, while also not requiring continuous rapid response of (touch) sensors. FIG. 9D shows a high-level process flow of this method.

Switching the delivery sensor off completely during the idle state reduces power consumption and the risk of accidental activation of the motion sensing unit. Alternatively, the delivery sensor may be left on in a slow response setting, which will, for example, allow for seamless pairing or synching functionality as described in conjunction with FIG. 9A. In the slow or low response rate setting, the delivery signal may still be used to initialize or activate the motion sensing unit. Therefore, the main remaining portion of an interrupted dose may still be covered (albeit with a delay as compared to an initialization starting from the delivery sensor in the high response rate mode). The delivery sensor is expediently configured to initialize or activate the motion sensing unit regardless of the response rate at which it is operated (e.g. for a manual synchronization or pairing process), of course.

Cancelling a synchronization event which might still be in progress in response to a new touch event of the setting surface can be avoided as the setting precedes the delivery and there is usually sufficient time to complete the synchronization event before the set dose is delivered and the system can distinguish which surface is being touched. If the delivery surface is touched, the event may be interrupted in order not to miss a dose delivery operation.

The setting surface may be more prone to detect accidentally occurring touch events owing to its circumferential disposition. Hence, using the delivery surface or sensor for activating the power consuming motion sensing unit is advantageous.

Having a comparatively short timeout for the motion sensing unit (e.g. less than 10 s, such as 3 to 5 s) may limit the power consumption in case of accidentally occurring touch events of the delivery surface (that is, if the delivery sensor is operational at all in the idle state). The idle state may be the standard state the system is in, e.g. after connecting the system to a device unit.

While the method as discussed above provides a sensible process flow which may stand alone, some variations may be used to further reduce the overall power consumption. Some variations are discussed below.

It has been observed that sensors, e.g. capacitive sensors, will generally draw significantly more than their quiescent current for the duration of touch or proximity events. In some applications the duration of the event or the point of release are of interest. This functionality may be sacrificed in order to increase the overall life of the electronic system, e.g. due to the power consumption being reduced.

For example, the system can be configured such that the setting sensor is switched off or disabled when an event has been detected by this sensor, e.g. a setting event. The setting sensor will detect a touch event at the start of a setting operation. The touch or contact will be maintained (intermittently) until the end of the setting operation, when the setting surface is released. However, after occurrence of the touch event, the setting sensor may provide no more useful information to the system. Therefore, from the point of the first touch onwards, the setting sensor can be disabled, e.g. until the electronic system returns to the first or idle state. If the touch is still present at the point of return to the idle state, the setting sensor may be immediately disabled once more and the process repeats. Depending on the type of touch or proximity sensor employed, it may be advantageous to negate the detected touch or proximity event of the setting surface before the setting sensor is disabled (otherwise a new touch event might not be detected when the sensor is re-enabled, e.g. in the idle state). This can be achieved by increasing the detection threshold to beyond the limits attainable by a physical touch and waiting until the sensor no longer registers a touch on the setting sensor before disabling it. The detection thresholds are expediently restored when the sensor is re-enabled or when the system enters the idle state.

Alternatively to switching the sensor off in response to the event, the setting sensor can be operated at a lower response rate, e.g. lower than in the first or idle state, after the event has been detected. The setting sensor may be switched to the lower response rate in response to the setting signal (i.e. in response to the touch or proximity event), in response to the delivery signal or in response to the (first) motion signal generated by the motion sensing unit.

Alternatively or additionally to reducing the power consumption of the setting sensor after the touch event is detected for the setting surface, the power consumption of the delivery sensor can be lowered after the touch event is detected, e.g. in response to the setting or delivery signal or in response to a motion signal generated by the motion sensing unit. In contrast to the setting sensor, it is not advisable to completely disable the delivery sensor after touch (especially not during “Dose capture”), as any potential power saving would be negated by no longer being able to detect the point of release. If the point of release cannot be detected, a potential signal would not be available to trigger exiting the dose capture procedure or inactivating the motion sensing unit. As explained previously, keeping the motion sensing unit powered unnecessarily is by far the greatest waste of electrical power. As a compromise, a reduced or low response rate, e.g. below 10 Hz, can be set for the delivery sensor, e.g. while the motion sensing unit is operational (with or without motion signals being generated) and/or when motion signals are generated. This is acceptable since the urgency of responding to a release is significantly less than that of responding to proximity or touch (as explained above slow response of the delivery sensor to proximity or touch may lead to an undercount of the dose).

The present disclosure provides features of an electronic system that can be embodied as a re-usable clip-on module with a suitably configured pen injector for the purpose of recording doses that are delivered from the pen. This functionality may be of value to a wide variety of users as a memory aid or to support detailed logging of dose history. The system can be configured to be electronically connectable to a mobile device, or similar, to enable the dose history to be downloaded from the system on a periodic basis.

The use of touch or proximity sensitivity, for example with a capacitive sensor, has the advantage of allowing the electronic system to be completely sealed from water ingress. A further advantage may be the protection against accidental activation in storage, as there will be no external moving parts involved in the activation, e.g. as compared to mechanical switches. This is particularly advantageous for the robustness of an electronic system which may be carried with a user for multiple years and subject to a variety of conditions.

In general, we note, that features which are disclosed herein in connection with different figures, aspects, embodiments and so on can also be combined with one another and also with features disclosed below and in the claims as well as in the drawings.

As has been discussed above, managing electrical power consumption or the resources of a power supply (e.g. a rechargeable or non-rechargeable battery) in drug delivery devices comprising electronic systems, such as the injection devices discussed further above, or systems for drug delivery devices is a problem which needs to be addressed, e.g. in order to optimize the use of the capacity of the power supply and/or in consideration of the sometimes considerable shelf time of a drug delivery device or an electronic system has before it reaches the user or patient. It needs to be ensured that the electronic system still functions properly for the duration of its intended use, even when storing the system for a longer period.

The present disclosure has presented various concepts which can be implemented in drug delivery devices or electronic systems thereof or therefore, e.g. for improving the power management in the devices. Some concepts rely on providing electrical power to certain units of the device only when needed or when it is very likely that the power will be needed. For example, the device which has been mentioned above and which is described in WO 2019/101962 A1, for example, energizes a motion sensing unit (the sensor system with IR-LEDs and IR-detectors) of the device only when the injection button (as user interface member) is being pressed for performing a dose delivery (injection) operation. The rotation of the encoder component or encoder ring can be used by the sensing unit to gather data on movements which are indicative for the dose which has been delivered during the delivery operation, after the motion sensing unit has been energized. From the measured movement data, it can be calculated, how much of the drug has actually been delivered. The amount of the actually delivered drug does not necessarily coincide with the dose which was previously set in a dose setting operation, e.g. when the user interrupts the delivery operation before it has been actually completed. Accordingly, it is advantageous to measure movements occurring during the dose delivery operation which are correlated to the amount of drug which has been delivered already, e.g. to get insight on the current status or the progress of the delivery operation. The determined delivered dose may be communicated, preferably wirelessly to an external or remote device, e.g. a hand-held device such as a smartphone, e.g. via the communication unit. In this way a dose log on the doses delivered by the user may be established, which may be accessed by the user easily.

The proposed concepts are suitable for a large variety of drug delivery devices comprising electronic systems or for electronic systems for such devices not only for the devices described further above. The device may be an injection device and/or a pen-type device. The device may be configured to receive or comprise a medicament container or cartridge. The container or cartridge may be filled with liquid drug to be delivered by the device. The device may be designed to deliver a plurality of doses of the drug. Consequently, the container or cartridge may comprise drug in amount sufficient for several doses to be delivered by the device. The device may be re-usable or disposable, where a re-usable device may be provided with a replacement medicament container or cartridge when the current container or cartridge is considered empty or needs to be replaced for different reasons. A disposable device may be a single use device which is disposed after the medicament container has been emptied. The device may be a device of a dial extension type, that is to say a device which increases in length during the dose setting operation, where the increase in length is proportional to the size of the set dose. During the associated dose delivery operation, the length of the device may be decreased again, e.g. until the device resumes its original length, i.e. the length it had before the dose setting operation has been commenced. Alternatively, the length of the device may be independent of the size of the set dose, e.g. constant or substantially constant during dose setting and/or dose delivery. The dose setting operation may involve a, preferably rotational, movement, of a dose setting member as user interface member, e.g. a knob, button or grip component (as discussed further above already). The dose delivery operation may involve a, preferably axial, movement of a dose delivery member as user interface member, e.g. a button such as the injection button discussed further above. As already discussed further above, the dose setting member and the dose delivery member may be formed by a single, e.g. unitary, component, where, preferably, different surfaces of the component are manipulated during the dose setting operation and the dose delivery operation or, alternatively, the dose setting member and the dose delivery member may be separate components/interface members or parts with relative movement being possible between these members, e.g. to switch the dose setting and drive mechanism between a dose setting configuration and a dose delivery configuration. There may be relative movement between these components either during dose setting or dose delivery or during both operations. During the dose setting operation a lateral or side surface, i.e. a radially facing surface, of the dose setting member may be gripped by the user, e.g. with the thumb and index finger. During the dose delivery operation an axially, e.g. proximally, facing surface of the dose delivery member may be touched by the user, e.g. with the thumb. During the dose delivery operation, an axial force may be transferred by the user to the dose delivery member in order to initiate and/or to continuously drive the dose delivery operation using a dose setting and drive mechanism of the device which aside, from the user interface member may comprise further members, such as a drive member and a piston rod, for example. The drive member may engage the piston rod. The drive member may be a drive sleeve. In one embodiment, the dose delivery member may be a drive member which engages the piston rod threadedly. The device may be a device as disclosed, for example, in WO 2015/028439 A1 the disclosure of which is incorporated herein by reference in its entirety. In this device, the knob/button may be rotationally locked with the dial sleeve or number sleeve during dose setting via an according clutch being engaged. When delivering the dose, the clutch is released and the knob is rotationally locked relative to the housing. The dial sleeve may rotated relative to the housing during dose delivery.

The device may be a needle-based device, i.e. the drug may be delivered into the body via a needle piercing the skin, or may be needle-free. The device may be a device with a delivery assist, e.g. a spring-assisted or spring-driven device. In such devices, the dose delivery operation by the user is assisted or entirely driven by energy provided by an energy storage member such as a spring. The energy in the storage member may be increased during the dose setting operation by the user or the energy storage member may be provided with the entire energy required to empty the medicament container pre-stored in the member by the manufacturer. In the latter case, the user does not need to provide energy to increase the energy stored in the energy storage member such as during the dose setting operation.

We note that the description of the embodiments focuses on the motion sensing unit gathering data during the dose delivery operation. It is, however, also possible to gather the data during dose setting. Moreover, the user proximity detection units which have been discussed are not only suitable for power management purposes but can also be used for other purposes, e.g. controlling the operation of or activating the communication unit, e.g. for transmitting and/or synchronizing dose data.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short-or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term, derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C, CM-3, GLP-1 Eligen, ORMD-0901, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, TT-401, BHM-034. MOD-6030, CAM-2036, DA-15864, ARI-2651, ARI-2255, Exenatide-XTEN and Glucagon-Xten.

An examples of an oligonucleotide is, for example: mipomersen sodium (Kynamro®), a cholesterol-reducing antisense therapeutic for the treatment of familial hypercholesterolemia. Examples of DPP4 inhibitors are Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

The scope of protection is not limited to the examples given herein above. Any invention disclosed herein is embodied in any novel characteristic and/or combination of characteristics, which particularly includes every combination of any features which are stated in the claims, even if this feature or this combination of features is not explicitly stated in the claims or in the examples.

### REFERENCE NUMERALS

1 injection device, drug delivery device or device unit
10 housing
12 dosage knob
11 injection button
13 window
14 container
15 needle
16 inner needle cap
17 outer needle cap
18 cap
70 sleeve
71a-c formation
1000 electronic system
1100 electronic control unit
1200 motion sensing unit
1300 use detection unit
1310 setting detection unit
1320 delivery detection unit
1330 user proximity detection unit
1340 sensor
1345 sensor electrode
1347 electrode portion
1348 ring portion
1349 connection portion
1350 wake-up unit
1400 communication unit
1500 power supply
1510 spacer component
1520 power supply electrode
1530 contact portion
1540 biasing portion
1550 securing portion
1560 ring portion
1570 supply contact portion
1600 user interface member
1605 user interface member body
1610 setting surface
1615 connection feature
1620 delivery surface
1630 contact surface
1640 contact surface
1650 delivery sensor
1660 setting sensor
1670 chassis
1672 rigid portion
1674 deformable portion
1676 light guide
1678 snap arm
1700 connection detection unit
1710 switch
3000 conductor carrier
4000 member
4010 connection
4020 recess
4030 interior
4040 contact portion
4050 central region
4060 edge region
4070 groove
4080 opening

1-29. (canceled)

30. An electronic system for a drug delivery device, the electronic system comprising:
at least one user interface member arranged to be manipulated by a user of the drug delivery device, wherein the user interface member has an exterior operation surface arranged and configured to be touched by the user of the drug delivery device during operation of the drug delivery device; and
an electrical user proximity detection unit configured to generate a signal when the user is close to or touches the exterior operation surface.

31. The electronic system of claim 30, wherein the exterior operation surface is or comprises a setting surface of the user interface member which is arranged to be contacted for conducting or initiating a dose setting operation, and wherein the exterior operation surface is the setting surface.

32. The electronic system of claim 30, wherein the user proximity detection unit comprises an electrical sensor or switch that is arranged and configured to detect whether the user is close to the exterior operation surface or touches the exterior operation surface, and wherein the sensor or switch is a contactless or touchless proximity sensor or switch, and/or wherein the user proximity detection unit comprises an electrical sensor or switch that is arranged and configured to detect whether the user touches the exterior operation surface, and wherein the sensor or switch is a contact sensor or switch.

33. The electronic system of claim 30, wherein the user proximity detection unit comprises a capacitive sensor or switch that is arranged and configured to detect whether the user is close to or touches the exterior operation surface.

34. The electronic system of claim 30, wherein the user proximity detection unit comprises a sensor or switch that comprises at least one electrically conductive contact surface accessible on the exterior operation surface, wherein the sensor or switch is arranged and configured to detect whether the user touches the exterior operation surface.

35. The electronic system of claim 30, wherein the user proximity detection unit comprises a sensor or switch that comprises multiple electrically conductive contact surfaces accessible on the exterior operation surface, wherein at least two electrically conductive contact surfaces of the multiple electrically conductive contact surfaces are arranged such that the user can simultaneously touch the at least two electrically conductive contact surfaces with one finger or two different fingers of a hand of the user, and
wherein the at least two electrically conductive contact surfaces are configured to be surfaces having different electrical potentials, wherein the electronic system is configured such that the signal is formed by a current flowing through the hand of the user from one of the electrically conductive contact surfaces to another one of the electrically conductive contact surfaces when the user touches both contact surfaces simultaneously.

36. The electronic system of claim 30, further comprising an electronic control unit configured to control an operation of the electronic system in response to the signal of the user proximity detection unit,
wherein the electronic system has a first state and a second state, wherein the electronic system has a higher electrical power consumption in the second state as compared to the first state,
wherein the electronic system further comprises an electrical use detection unit operatively connected to the electronic control unit, wherein the electrical use detection unit is configured to generate a use signal that indicates the user intends to perform a dose setting operation and/or a dose delivery operation, wherein the electronic system is configured to be switched from the first state into the second state in response to the use signal,
wherein the use detection unit comprises the user proximity detection unit, and
wherein the use signal is the signal generated by the user proximity detection unit when the user is close to or touches the exterior operation surface.

37. The electronic system of claim 36, wherein the electronic system comprises a motion sensing unit (1200),
wherein the motion sensing unit is configured to generate electrical motion signals suitable to quantify a relative movement of a first member relative to a second member,
wherein the motion sensing unit is configured to be switched by the electronic control unit from a first state, in which the motion sensing unit is not operative, into a second state, in which the motion sensing unit is operative, in response to the use signal, and
wherein the electronic system is configured such that the motion sensing unit operates during the dose delivery operation.

38. The electronic system of claim 37,
wherein the exterior operation surface is or comprises a setting surface of the user interface member arranged to be contacted for conducting or initiating a dose setting operation, and wherein the exterior operation surface associated with generating the use signal is the setting surface, and the motion sensing unit is configured to monitor the dose delivery operation.

39. The electronic system of claim 37,
wherein the exterior operation surface is or comprises a delivery surface arranged to be contacted for conducting or initiating a dose delivery operation, and wherein the exterior operation surface associated with generating the use signal is the delivery surface, and the motion sensing unit is configured to monitor the dose delivery operation.

40. The electronic system of claim 30, further comprising a wake-up unit configured to generate an electrical wake-up signal, wherein the electronic system is configured to switch the user proximity detection unit from a first state to a second state of higher power consumption in response to the wake-up signal,
wherein the wake-up unit comprises an electrical wake-up sensor or switch configured to generate the electrical wake-up signal.

41. The electronic system of claim 40, wherein the electrical wake-up sensor or switch is a vibration sensor or switch, a capacitive sensor or switch, or a resistive sensor or switch.

42. The electronic system of claim 30, wherein the electronic system is configured as a reusable add-on system for a disposable drug delivery device unit.

43. The electronic system of claim 30, further comprising at least one sensor or switch configured to be deactivated in response to the signal generated by the user proximity detection unit.

44. The electronic system of claim 43, wherein the at least one sensor or switch which is deactivated is the same sensor or switch generating the signal of the user proximity detection unit in response to which it is deactivated.

45. The electronic system of claim 30, further comprising at least one sensor or switch configured to have a response rate that changes in response to the signal generated by the user proximity detection unit,
wherein the response rate of the at least one sensor or switch is increased or decreased in response to the signal generated by the user proximity detection unit.

46. The electronic system of claim 30, further comprising at least one sensor or switch configured to be deactivated or to change its response rate in response to the signal generated by the user proximity detection unit, and
wherein the at least one sensor or switch is different from a sensor or switch that generates the signal of the proximity detection unit.

47. The electronic system of claim 30, further comprising at least one sensor or switch configured to be deactivated or to change its response rate in response to the signal generated by the user proximity detection unit,
wherein the user proximity detection unit comprises the at least one sensor or switch,
wherein the at least one sensor or switch is the same as a sensor or switch that generates the signal of the user proximity detection unit.

48. A drug delivery device comprising:
an electronic system that comprises
at least one user interface member arranged to be manipulated by a user of the drug delivery device, wherein the user interface member has an exterior operation surface arranged and configured to be touched by the user of the drug delivery device during operation of the drug delivery device; and
an electrical user proximity detection unit configured to generate a signal when the user is close to or touches the exterior operation surface,
wherein the drug delivery device comprises a reservoir with drug.

49. The drug delivery device of claim 48, wherein the drug delivery device is configured such that the device increases its length during a dose setting operation in an amount proportional to a size of a set dose.
